(19) United States
(12) Patent Application Publication
(10) Pub. No.: US 2013/0023531 A1
(43) Pub. Date: Jan. 24, 2013

(54) PYRIMIDO[5,4-D]PYRIMIDYLAMINO PHENYL SULFONAMIDES AS SERINE/THREONINE KINASE INHIBITORS

(71) Applicants: Andreas MANTOULIDIS, Vienna (AT); Peter ETTMAYER, Vienna (AT)

(72) Inventors: Andreas MANTOULIDIS, Vienna (AT); Peter ETTMAYER, Vienna (AT)

(73) Assignee: BOEHRINGER INGELHEIM INTERNATIONAL GMBH, Ingelheim am Rhein (DE)

(21) Appl. No.: 13/353,402

(22) Filed: Jan. 19, 2012

(30) Foreign Application Priority Data
Jan. 27, 2011 (EP) 11152366.8

(51) Int. Cl. A61K 31/519; A61P 35/00; C07D 471/10; C07D 487/04; A61K 31/5377

(52) U.S. Cl. 514/234.2; 544/256; 514/262.1; 544/118; 514/252.16; 544/230

(57) ABSTRACT

The present invention encompasses compounds of general formula (I)

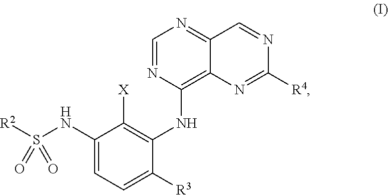

wherein the groups R² to R⁴ and X are defined as in claim 1, which are suitable for the treatment of diseases characterised by excessive or abnormal cell proliferation, pharmaceutical preparations which contain compounds of this kind and their use as medicaments.

The present invention relates to new pyrimido[5,4-d]pyrimidylamino phenyl sulfonamides of general formula (I)

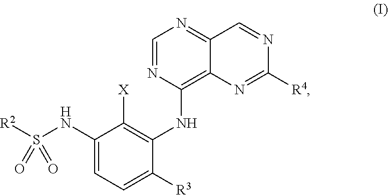

wherein the groups R² to R⁴ and X have the meanings given in the claims and specification, pharmaceutical preparations which contain compounds of this kind and their use as medicaments.

### BACKGROUND TO THE INVENTION

Various fluorine-substituted phenyl sulfonamides are described in WO 2009/012283 as modulators of various kinases.

The aim of the present invention is to indicate new pyrimido[5,4-d]pyrimidylamino phenyl sulfonamides which may be used for the prevention and/or treatment of diseases characterised by excessive or abnormal cell proliferation. The pyrimido[5,4-d]pyrimidylamino phenyl sulfonamides according to the invention are distinguished by their great inhibitory effect on B-Raf V600E and their improved high potency against tumour cells, e.g. melanoma cells, which is achieved by the selective inhibition of B-Raf V600E and can also be demonstrated in vivo. Apart from the inhibitory effect and the cell potency the compounds additionally have good pharmacokinetic properties. As a result of this overall profile, the compounds according to the invention are suitable for the development of a drug.

The RAS-RAF-MAPK (mitogen-activated protein kinase) signaling pathway plays a critical role in transmitting proliferation signals generated by the cell surface receptors and cytoplasmic signaling elements to the nucleus. Constitutive activation of this pathway is involved in malignant transformation by several oncogenes. Activating mutations in RAS occur in approximately 15% of cancers, and recent data has shown that B-RAF is mutated in about 7% of cancers (Wellbrock et al., Nature Rev. Mol. Cell. Biol. 2004, 5:875-885), identifying it as another important oncogene in this pathway. In mammals, the RAF family of serine/threonine kinases comprises three members: A-RAF, B-RAF and C-RAF. However, activating mutations have so far been only identified in B-RAF underlining the importance of this isoform. It is believed that B-RAF is the main isoform that couples RAS to MEK, and that C-RAF and A-RAF signal to ERK only to fine-tune cellular responses (Wellbrock et al., Nature Rev. Mol. Cell. Biol. 2004, 5:875-885). The most common cancer mutation in B-RAF results in a valine to glutamic acid exchange at position 600 of the protein (V600E), which dramatically enhances B-RAF activity, presumably because its negative charge mimics activation loop phosphorylation (Wan et al., Cell 2004, 116: 855-867). The highest incidence of B-RAF V600 mutations occurs in malignant melanoma (38%), thyroid cancer (38%), colorectal cancer (10%), bilary tract cancer (12%) and ovarian cancer (12%), but they also occur at a low frequency in a wide variety of other cancers (frequencies of mutations according to COSMIC (Catalogue Of Somatic Mutations In Cancer; Wellcome Trust Sanger Institute) release v49, 29 Sep. 2010). Literature supported the hypothesis that B-RAF^{V600E} mutated tumour cells seem to rely heavily on the continued activation of this pathway—a phenomenon termed “oncogene addiction”—whereas normal B-RAF^{wt} cells use a broader range of signals. This provides an Achilles' heel that can be exploited therapeutically by treating patients with somatically mutated B-RAF^{V600E} using orally available B-RAF inhibitors.

The key role of B-RAF^{V600E} in aberrant ERK signaling and consequently oncogenesis has been demonstrated in several independent experimental approaches such as overexpression of oncogenic/mutated B-RAF in vitro and in vivo (Wan et al., Cell 2004, 116: 855-867; Wellbrock et al., Cancer Res. 2004, 64: 2338-2342), siRNA knock-down in vitro (Karasarides et al., Oncogene 2004, 23: 6292-6298) or in inducible short-hairpin RNA xenograft models where gain-of-function B-RAF signaling was found to be strongly associated with in vivo tumorigenicity (Hoeflich et al., Cancer Res. 2006, 66: 999-1006).

Treatment of B-RAF^{V600E} mutated melanoma or colon carcinoma cells induces a B-RAF inhibition phenotype (e.g. reduction of phospho-MEK and phospho-ERK levels, reduction of cyclin D expression and induction of p27 expression). Consequently, these cells are locked in the G1-phase of the cell cycle and do not proliferate.

### DETAILED DESCRIPTION OF THE INVENTION

It has now been found that, surprisingly, compounds of general formula (I) wherein the groups R² to R⁴ and X have the meanings given hereinafter act as inhibitors of specific signal enzymes which are involved in controlling cell proliferation. Thus, the compounds according to the invention may be used for example for the treatment of diseases connected with the activity of these signal enzymes and characterised by excessive or abnormal cell proliferation.

The present invention therefore relates to compounds of general formula (I)

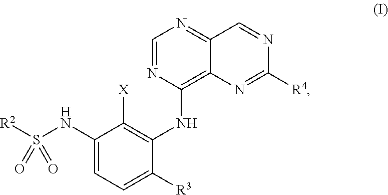

wherein

### (A0)

R² is a group optionally substituted by one or more, identical or different R^{b1} and/or R^{c1}, selected from among C₁₋₆alkyl, C₂₋₆alkenyl, C₂₋₆alkynyl, C₁₋₆haloalkyl, C₃₋₆cycloalkyl, C₄₋₆cycloalkenyl, C₆₋₁₀aryl, 5-10 membered heteroaryl and 3-10 membered heterocyclyl or

R² is —NR^{c1}R^{c1};
each R^{b1} is independently selected from among —OR^{c1}, —NR^{c1}R^{c1}, halogen, —CN, —C(O)R^{c1}, —C(O)OR^{c1}, —C(O)NR^{c1}R^{c1}, —S(O)₂R^{c1}, —NHC(O)R^{c1} and —N(C₁₋₄alkyl)C(O)R^{c1} as well as the bivalent substituent ═O, wherein the latter may only be a substituent in non-aromatic ring systems;

each R^{c1} independently of one another denotes hydrogen or a group selected from among C₁₋₆alkyl, C₂₋₆alkenyl, C₂₋₆alkynyl, C₁₋₆haloalkyl, C₃₋₆cycloalkyl, C₄₋₆cycloalkenyl, C₆₋₁₀aryl, 5-10 membered heteroaryl and 3-10 membered heterocyclyl;

### (B0)

R³ is selected from among hydrogen, halogen, C₁₋₄alkyl, C₁₋₄alkyloxy, C₂₋₄alkenyl, C₂₋₄alkynyl, C₁₋₄haloalkyl, —CN, —NH(C₁₋₄alkyl) and —N(C₁₋₄alkyl)₂;

### (C0)

R⁴ denotes a group optionally substituted by one or more, identical or different R^{a2} and/or R^{b2}, selected from among C₁₋₆alkyl, C₂₋₆alkenyl, C₂₋₆alkynyl, C₁₋₆haloalkyl, C₃₋₆cycloalkyl, C₄₋₆cycloalkenyl, C₆₋₁₀aryl, 5-10 membered heteroaryl and 3-11 membered heterocyclyl, or is selected from among —OR^{a3}, —NR^{a3}R^{a3}, —N(OR^{a3})R^{a3}, —CN, —C(O)R^{a3}, —C(O)OR^{a3}, —C(O)NR^{a3}R^{a3}, —C(NH)NR^{a3}R^{a3}, —S(O)₂NR^{a3}R^{a3}, —NHS(O)₂R^{a3}, —N(C₁₋₄alkyl)S(O)₂R^{a3}, —NHS(O)₂NR^{a3}R^{a3}, —NHC(O)R^{a3}, —N(C₁₋₄alkyl)C(O)R^{a3}, —NHC(O)OR^{a3}, —N(C₁₋₄alkyl)C(O)OR^{a3}, —NHC(O)NR^{a3}R^{a3} and —N(C₁-4alkyl)C(O)NR^{a3}R^{a3};
each R^{a2} independently of one another denotes a group optionally substituted by one or more, identical or different R^{b2} and/or R^{c2}, selected from among C₁₋₆alkyl, C₂₋₆alkenyl, C₂₋₆alkynyl, C₁₋₆haloalkyl, C₃₋₆cycloalkyl, C₄₋₆cycloalkenyl and 3-10 membered heterocyclyl;
each R^{b2} is independently selected from among —OR^{c2}, —NR^{c2}R^{c2}, halogen, —C(O)R^{c2}, —C(O)OR^{c2}, —C(O)NR^{c2}R^{c2}, —CN, —NHC(O)R^{c2} and —NHC(O)OR^{c2};
each R^{c2} independently of one another denotes hydrogen or a group selected from among C₁₋₆alkyl, C₂₋₆alkenyl, C₂₋₆alkynyl, C₃₋₆cycloalkyl, C₄₋₆cycloalkenyl and 3-10 membered heterocyclyl, wherein this heterocyclyl is optionally substituted by one or more, identical or different substituents selected from among halogen, C₁₋₆alkyl and —C(O)—C₁₋₆alkyl;
each R^{a3} independently of one another denotes hydrogen or a group optionally substituted by one or more, identical or different R^{b3} and/or R^{c3}, selected from among C₁₋₆alkyl, C₂₋₆alkenyl, C₂₋₆alkynyl, C₁₋₆haloalkyl, C₃₋₆cycloalkyl, C₄₋₆cycloalkenyl, C₆₋₁₀aryl, 5-10 membered heteroaryl and 3-10 membered heterocyclyl;
each R^{b3} is independently selected from among —OR^{c3}, —NR^{c3}R^{c3}, halogen, —C(O)R^{c3}, —C(O)OR^{c3}, —C(O)NR^{c3}R^{c3}, —CN, —NHC(O)R^{c3} and —NHC(O)OR^{c3};
each R^{c3} independently of one another denotes hydrogen or a group selected from among C₁₋₆alkyl, C₂₋₆alkenyl, C₂₋₆alkynyl, C₃₋₆cycloalkyl, C₄₋₆cycloalkenyl, C₁₋₆alkyl-O—C₁₋₆alkyl, (C₁₋₄alkyl)HN—C₁₋₆alkyl, (C₁₋₄alkyl)₂N—C₁₋₆alkyl, C₁₋₆haloalkyl, 4-16 membered heterocyclylalkyl and 3-10 membered heterocyclyl, wherein the heterocyclyl ring in aforementioned groups is optionally substituted by one or more, identical or different C₁₋₆alkyl;

### (D0)

X denotes chlorine or fluorine;

wherein the compounds (I) may optionally also be present in the form of the tautomers, racemates, enantiomers, diastereomers and the mixtures thereof or as the respective salts of all the above-mentioned forms.

In one aspect (A1) the invention relates to compounds (I), wherein

R² denotes C₁₋₆alkyl or phenyl substituted with one or more, identical or different halogen.

In another aspect (A2) the invention relates to compounds (I), wherein

R² is selected from among ethyl, n-propyl, iso-propyl and iso-butyl.

In another aspect (A3) the invention relates to compounds (I), wherein

R² is n-propyl.

In another aspect (B1) the invention relates to compounds (I), wherein

R³ is halogen.

In another aspect (B2) the invention relates to compounds (I), wherein

R³ is fluorine.

In another aspect (C1) the invention relates to compounds (I), wherein

R⁴ is 3-11 membered heterocyclyl optionally substituted by one or more, identical or different R^{a2} and/or R^{b2}
each R^{a2} independently of one another denotes a group optionally substituted by one or more, identical or different R^{b2} and/or R^{c2}, selected from among C₁₋₆alkyl, C₂₋₆alkenyl, C₂₋₆alkynyl, C₁₋₆haloalkyl, C₃₋₆cycloalkyl, C₄₋₆cycloalkenyl and 3-10 membered heterocyclyl;
each R^{b2} is independently selected from among —OR^{c2}, —NR^{c2}R^{c2}, halogen, —C(O)R^{c2}, —C(O)OR^{c2}, —C(O)NR^{c2}R^{c2}, —CN, —NHC(O)R^{c2} and —NHC(O)OR^{c2}, and
each R^{c2} independently of one another denotes hydrogen or a group selected from among C₁₋₆alkyl, C₂₋₆alkenyl, C₂₋₆alkynyl, C₃₋₆cycloalkyl, C₄₋₆cycloalkenyl and 3-10 membered heterocyclyl, wherein this heterocyclyl is optionally substituted by one or more, identical or different substituents selected from among halogen, C₁₋₆alkyl and —C(O)—C₁₋₆alkyl.

In another aspect (C2) the invention relates to compounds (I), wherein

R⁴ is 4-7 membered, nitrogen-containing heterocyclyl optionally substituted by one or more, identical or different R^{a2} and/or R^{b2}
each R^{a2} independently of one another denotes a group optionally substituted by one or more, identical or different R^{b2} and/or R^{c2}, selected from among C₁₋₆alkyl, C₂₋₆alkenyl, C₂₋₆alkynyl, C₁₋₆haloalkyl, C₃₋₆cycloalkyl, C₄₋₆cycloalkenyl and 3-10 membered heterocyclyl;
each R^{b2} is independently selected from among —OR^{c1}, —NR^{c2}R^{c2}, halogen, —C(O)R^{c2}, —C(O)OR^{c2}, —C(O)NR^{c2}R^{c2}, —CN, —NHC(O)R^{c2} and —NHC(O)OR^{c2}, and
each R^{c2} independently of one another denotes hydrogen or a group selected from among C₁₋₆alkyl, C₂₋₆alkenyl, C₂₋₆alkynyl, C₃₋₆cycloalkyl, C₄₋₆cycloalkenyl and 3-10 membered heterocyclyl, wherein this heterocyclyl is optionally substituted by one or more, identical or different substituents selected from among halogen, C₁₋₆alkyl and —C(O)—C₁₋₆alkyl.

In another aspect (C3) the invention relates to compounds (I), wherein

R⁴ is selected from among piperazinyl, piperidinyl, pyrrolidinyl and morpholinyl, all optionally substituted by one or more, identical or different R^{a2} and/or R^{b2}
each R^{a2} independently of one another denotes a group optionally substituted by one or more, identical or different R^{b2} and/or R^{c2}, selected from among C₁₋₆alkyl, C₂₋₆alkenyl, C₂₋₆alkynyl, C₁₋₆haloalkyl, C₃₋₆cycloalkyl, C₄₋₆cycloalkenyl and 3-10 membered heterocyclyl;
each R^{b2} is independently selected from among —OR^{c2}, —NR^{c2}R^{c2}, halogen, —C(O)R^{c2}, —C(O)OR^{c2}, —C(O)NR^{c2}R^{c2}, —CN, —NHC(O)R^{c2} and —NHC(O)OR^{c2}, and
each R^{c2} independently of one another denotes hydrogen or a group selected from among C₁₋₆alkyl, C₂₋₆alkenyl, C₂₋₆alkynyl, C₃₋₆cycloalkyl, C₄₋₆cycloalkenyl and 3-10 membered heterocyclyl, wherein this heterocyclyl is optionally substituted by one or more, identical or different substituents selected from among halogen, C₁₋₆alkyl and —C(O)—C₁₋₆alkyl.

In another aspect (C4) the invention relates to compounds (I), wherein

R⁴ is selected from among piperazinyl, piperidinyl, pyrrolidinyl and morpholinyl, all bound to the pyrimido[5,4-d]pyrimidine ring system via a nitrogen atom and all optionally substituted by one or more, identical or different R^{a2} and/or R^{b2}
each R^{a2} independently of one another denotes a group optionally substituted by one or more, identical or different R^{b2} and/or R^{c2}, selected from among C₁₋₆alkyl, C₂₋₆alkenyl, C₂₋₆alkynyl, C₁₋₆haloalkyl, C₃₋₆cycloalkyl, C₄₋₆cycloalkenyl and 3-10 membered heterocyclyl;
each R^{b2} is independently selected from among —OR^{c2}, —NR^{c2}R^{c2}, halogen, —C(O)R^{c2}, —C(O)OR^{c2}, —C(O)NR^{c2}R^{c2}, —CN, —NHC(O)R^{c2} and —NHC(O)OR^{c2}, and
each R^{c2} independently of one another denotes hydrogen or a group selected from among C₁₋₆alkyl, C₂₋₆alkenyl, C₂₋₆alkynyl, C₃₋₆cycloalkyl, C₄₋₆cycloalkenyl and 3-10 membered heterocyclyl, wherein this heterocyclyl is optionally substituted by one or more, identical or different substituents selected from among halogen, C₁₋₆alkyl and —C(O)—C₁₋₆alkyl.

In further aspects (C5)(C6)(C7)(C8) the invention relates to compounds (I) with structural aspects (C1)(C2)(C3)(C4), wherein
each R^{a2} independently of one another denotes a group optionally substituted by one or more, identical or different R^{b2} and/or R^{c2}, selected from among C₁₋₆alkyl, C₃₋₆cycloalkyl and 3-10 membered heterocyclyl;
each R^{b2} is independently selected from among —OR^{c2}, —NR^{c2}R^{c2} and halogen, and
each R^{c2} independently of one another denotes hydrogen or a group selected from among C₁₋₆alkyl, C₃₋₆cycloalkyl and 3-10 membered heterocyclyl, wherein this heterocyclyl is optionally substituted by one or more, identical or different substituents selected from among halogen, C₁₋₆alkyl and —C(O)—C₁₋₆alkyl.

In another aspect (C9) the invention relates to compounds (I), wherein

R⁴ is —NR^{a3}R^{a3};
each R^{a3} independently of one another denotes hydrogen or a group optionally substituted by one or more, identical or different R^{b3} and/or R^{c3}, selected from among C₁₋₆alkyl, C₂₋₆alkenyl, C₂₋₆alkynyl, C₁₋₆haloalkyl, C₃₋₆cycloalkyl, C₄₋₆cycloalkenyl, C₆₋₁₀aryl, 5-10 membered heteroaryl and 3-10 membered heterocyclyl;
each R^{b3} is independently selected from among —OR^{c3}, —NR^{c3}R^{c3}, halogen, —C(O)R^{c3}, —C(O)OR^{c3}, —C(O)NR^{c3}R^{c3}, —CN, —NHC(O)R^{c3} and —NHC(O)OR^{c3};

each R^{c3} independently of one another denotes hydrogen or a group selected from among C₁₋₆alkyl, C₂₋₆alkenyl, C₂₋₆alkynyl, C₃₋₆cycloalkyl, C₄₋₆cycloalkenyl, C₁₋₆alkyl-O—C₁₋₆alkyl, (C₁₋₄alkyl)HN—C₁₋₆alkyl, (C₁₋₄alkyl)₂N—C₁₋₆alkyl, C₁₋₆haloalkyl, 4-16 membered heterocyclylalkyl and 3-10 membered heterocyclyl, wherein the heterocyclyl ring in aforementioned groups is optionally substituted by one or more, identical or different C₁₋₆alkyl;

In another aspect (C10) the invention relates to compounds (I), wherein

R⁴ is —NR⁵R⁶;
R⁵ is hydrogen and
R⁶ is selected from among C₃₋₆cycloalkyl, 3-7 membered heterocyclyl, phenyl and pyridyl, all optionally substituted by one or two, identical or different substituents selected from among C₁₋₆alkyl, C₁₋₆alkyloxy-C₁₋₆alkyl and 3-7 membered heterocyclyl, wherein said last mentioned 3-7 membered heterocyclyl is optionally substituted by C₁₋₆alkyl.

In another aspect (C11) the invention relates to compounds (I), wherein

R⁴ is 5-6 membered, nitrogen-containing heteroaryl, optionally substituted by one, two or three, identical or different C₁₋₄alkyl.

In another aspect (C12) the invention relates to compounds (I), wherein

R⁴ is 5-membered, nitrogen-containing heteroaryl bound to the pyrimido[5,4-d]pyrimidine ring system via a nitrogen atom and optionally substituted by one, two or three, identical or different C₁₋₄alkyl.

In another aspect (C13) the invention relates to compounds (I), wherein

R⁴ is imidazolyl bound to the pyrimido[5,4-d]pyrimidine ring system via a nitrogen atom and optionally substituted by one, two or three, identical or different C₁₋₄alkyl.

In another aspect (D1) the invention relates to compounds (I), wherein

X denotes chlorine.

In another aspect (D2) the invention relates to compounds (I), wherein

X denotes fluorine.

All the above-mentioned structural aspects A1 to A3, B1 and B2, C1 to C13 and D1 and D2 are preferred embodiments of the various aspects A0, B0, C0 and D0 respectively. The structural aspects A0 to A3, B0 to B2, C0 to C13 and D0 to D2 relating to different molecular parts of the compounds (I) according to the invention may be permutated with one another as desired in combinations ABCD, so as to obtain preferred compounds (I). Each combination ABCD represents and defines individual embodiments or generic amounts of compounds according to the invention. Each individual embodiment or partial quantity defined by this combination is expressly also included and is a subject of the invention.

In another aspect the invention relates to intermediate compounds of general formula (II)

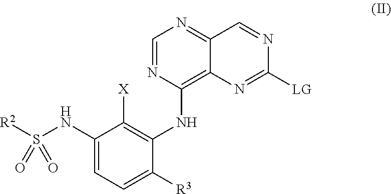

which can be used as precursors in the synthesis of compounds (I) according to the invention, wherein

R², R³ and X are as defined herein above, i.e. according to aspect A0, B0 and D0, or according to any of above given preferred embodiments A1 to A3, B1 and B2 and D1 and D2 including every combination of A0 to A3 with B0 to B2 and D0 to D2, and

LG is a suitable leaving group removable in nucleophilic substitution reactions as known by the skilled person, preferably selected from halogen, triflate, C₁₋₆alkyl-S—, C₁₋₆alkyl-S(O)— and C₁₋₆alkyl-S(O)₂—, more preferably selected from fluorine, chlorine, bromine, iodine, Me-S—, Me-S(O)— and Me-S(O)₂—.

The present invention further relates to hydrates, solvates, polymorphs, metabolites, derivatives and prodrugs of compounds of general formula (I).

The present invention further relates to a pharmaceutically acceptable salt of a compound of general formula (I) with anorganic or organic acids or bases.

In another aspect the invention relates to compounds of general formula (I)—or the pharmaceutically acceptable salts thereof—as medicaments.

In another aspect the invention relates to compounds of general formula (I)—or the pharmaceutically acceptable salts thereof—for use in a method for treatment of the human or animal body.

In another aspect the invention relates to compounds of general formula (I)—or the pharmaceutically acceptable salts thereof—for use in the treatment and/or prevention of cancer, infections, inflammations and autoimmune diseases.

In another aspect the invention relates to compounds of general formula (I)—or the pharmaceutically acceptable salts thereof—for use in a method for treatment and/or prevention of cancer, infections, inflammations and autoimmune diseases in the human and animal body.

In another aspect the invention relates to the use of compounds of general formula (I)—or the pharmaceutically acceptable salts thereof—for preparing a pharmaceutical composition for the treatment and/or prevention of cancer, infections, inflammations and autoimmune diseases.

In another aspect the invention relates to compounds of general formula (I)—or the pharmaceutically acceptable salts thereof—for use in the treatment and/or prevention of cancer.

In another aspect the invention relates to the use of compounds of general formula (I)—or the pharmaceutically acceptable salts thereof—for preparing a pharmaceutical composition for the treatment and/or prevention of cancer.

In another aspect the invention relates to compounds of general formula (I)—or the pharmaceutically acceptable salts thereof—for use in a method for treatment and/or prevention of cancer in the human or animal body.

In another aspect the invention relates to compounds of general formula (I)—or the pharmaceutically acceptable salts thereof—for use in the treatment and/or prevention of colon carcinomas, melanomas, cancer of the gall bladder and thyroid carcinomas.

In another aspect the invention relates to the use of compounds of general formula (I)—or the pharmaceutically acceptable salts thereof—for preparing a pharmaceutical composition for the treatment and/or prevention of colon carcinomas, melanomas, cancer of the gall bladder and thyroid carcinomas.

In another aspect the invention relates to a process for the treatment and/or prevention of cancer comprising administering a therapeutically effective amount of a compound of general formula (I)—or one of the pharmaceutically acceptable salts thereof—to a human being.

In another aspect the invention relates to a pharmaceutical preparation containing as active substance one or more compounds of general formula (I)—or the pharmaceutically acceptable salts thereof—optionally in combination with conventional excipients and/or carriers.

In another aspect the invention relates to a pharmaceutical preparation comprising a compound of general formula (I)—or one of the pharmaceutically acceptable salts thereof—and at least one other cytostatic or cytotoxic active substance, different from formula (I).

### DEFINITIONS

Terms that are not specifically defined here have the meanings that are apparent to the skilled man in the light of the overall disclosure and the context as a whole.

As used herein, the following definitions apply, unless stated otherwise:

The use of the prefix C_{x-y}, wherein x and y each represent a natural number (x<y), indicates that the chain or ring structure or combination of chain and ring structure as a whole, specified and mentioned in direct association, may consist of a maximum of y and a minimum of x carbon atoms.

The indication of the number of members in groups that contain one or more heteroatom(s) (heteroalkyl, heteroaryl, heteroarylalkyl, heterocyclyl, heterocycylalkyl) relates to the total number of atoms of all the ring members or chain members or the total of all the ring and chain members.

The indication of the number of carbon atoms in groups that consist of a combination of carbon chain and carbon ring structure (cycloalkylalkyl, arylalkyl) relates to the total number of carbon atoms of all the carbon ring and carbon chain members.

Alkyl denotes monovalent, saturated hydrocarbon chains, which may be present in both straight-chain (unbranched) and branched form. If an alkyl is substituted, the substitution may take place independently of one another, by mono- or polysubstitution in each case, on all the hydrogen-carrying carbon atoms.

The term “C₁₋₅alkyl” includes for example H₃C—, H₃C—CH₂—, H₃C—CH₂—CH₂—, H₃—CH(CH₃)—, H₃C—CH₂—CH₂—CH₂—, H₃C—CH₂—CH(CH₃)—, H₃C—CH(CH₃)—CH₂—, H₃C—C(CH₃)₂—, H₃C—CH₂—CH₂—CH₂—CH₂—, H₃C—CH₂—CH₂—CH(CH₃)—, H₃C—CH₂—CH(CH₃)—CH₂—, H₃C—CH(CH₃)—CH₂—CH₂—, H₃C—CH₂—C(CH₃)₂—, H₃C—C(CH₃)₂—CH₂—, H₃C—CH(CH₃)—CH(CH₃)— and H₃C—CH₂—CH(CH₂CH₃)—.

Further examples of alkyl are methyl (Me; —CH₃), ethyl (Et; —CH₂CH₃), 1-propyl (n-propyl; n-Pr; —CH₂CH₂CH₃), 2-propyl (i-Pr; iso-propyl; —CH(CH₃)₂), 1-butyl (n-butyl; n-Bu; —CH₂CH₂CH₂CH₃), 2-methyl-1-propyl (iso-butyl; i-Bu; —CH₂CH(CH₃)₂), 2-butyl (sec-butyl; sec-Bu; —CH(CH₃)CH₂CH₃), 2-methyl-2-propyl (tert-butyl; t-Bu; —C(CH₃)₃), 1-pentyl (n-pentyl; —CH₂CH₂CH₂CH₂CH₃), 2-pentyl (—CH(CH₃)CH₂CH₂CH₃), 3-pentyl (—CH(CH₂CH₃)₂), 3-methyl-1-butyl (iso-pentyl; —CH₂CH₂CH(CH₃)₂), 2-methyl-2-butyl (—C(CH₃)₂CH₂CH₃), 3-methyl-2-butyl (—CH(CH₃)CH(CH₃)₂), 2,2-dimethyl-1-propyl (neo-pentyl; —CH₂C(CH₃)₃), 2-methyl-1-butyl (—CH₂CH(CH₃)CH₂CH₃), 1-hexyl (n-hexyl; —CH₂CH₂CH₂CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂), 3,3-dimethyl-2-butyl (—CH(CH₃)C(CH₃)₃), 2,3-dimethyl-1-butyl (—CH₂CH(CH₃)CH(CH₃)CH₃), 2,2-dimethyl-1-butyl (—CH₂C(CH₃)₂CH₂CH₃), 3,3-dimethyl-1-butyl (—CH₂CH₂C(CH₃)₃), 2-methyl-1-pentyl (—CH₂CH(CH₃)CH₂CH₂CH₃), 3-methyl-1-pentyl (—CH₂CH₂CH(CH₃)CH₂CH₃), 1-heptyl (n-heptyl), 2-methyl-1-hexyl, 3-methyl-1-hexyl, 2,2-dimethyl-1-pentyl, 2,3-dimethyl-1-pentyl, 2,4-dimethyl-1-pentyl, 3,3-dimethyl-1-pentyl, 2,2,3-trimethyl-1-butyl, 3-ethyl-1-pentyl, 1-octyl (n-octyl), 1-nonyl (n-nonyl); 1-decyl (n-decyl) etc.

By the terms propyl, butyl, pentyl, hexyl, heptyl, octyl, nonyl, decyl etc. without any further definition are meant saturated hydrocarbon groups with the corresponding number of carbon atoms, wherein all isomeric forms are included.

The above definition for alkyl also applies if alkyl is a part of another (combined) group such as for example C_{x-y}alkylamino or C_{x-y}alkyloxy.

The term alkylene can also be derived from alkyl. Alkylene is bivalent, unlike alkyl, and requires two binding partners. Formally, the second valency is produced by removing a hydrogen atom in an alkyl. Corresponding groups are for example —CH₃ and —CH₂—, —CH₂CH₃ and —CH₂CH₂— or >CHCH₃ etc.

The term “C₁₋₄alkylene” includes for example —(CH₂)—, —(CH₂—CH₂)—, —(CH(CH₃))—, —(CH₂—CH₂—CH₂)—, —(C(CH₃)₂)—, —(CH(CH₂CH₃))—, —(CH(CH₃)—CH₂)—, —(CH₂—CH(CH₃))—, —(CH₂—CH₂—CH₂—CH₂)—, —(CH₂—CH₂—CH(CH₃))—, —(CH(CH₃)—CH₂—CH₂)—, —(CH₂—CH(CH₃)—CH₂)—, —(CH₂—C(CH₃)₂)—, —(C(CH₃)₂—CH₂)—, —(CH(CH₃)—CH(CH₃))—, —(CH₂—CH(CH₂CH₃))—, —(CH(CH₂CH₃)—CH₂)—, —(CH(CH₂CH₂CH₃))—, —(CHCH(CH₃)₂)— and —C(CH₃)(CH₂CH₃)—.

Other examples of alkylene are methylene, ethylene, propylene, 1-methylethylene, butylene, 1-methylpropylene, 1,1-dimethylethylene, 1,2-dimethylethylene, pentylene, 1,1-dimethylpropylene, 2,2-dimethylpropylene, 1,2-dimethylpropylene, 1,3-dimethylpropylene, hexylene etc.

By the generic terms propylene, butylene, pentylene, hexylene etc. without any further definition are meant all the conceivable isomeric forms with the corresponding number of carbon atoms, i.e. propylene includes 1-methylethylene and butylene includes 1-methylpropylene, 2-methylpropylene, 1,1-dimethylethylene and 1,2-dimethylethylene. The above definition for alkylene also applies if alkylene is part of another (combined) group such as for example in HO—C_{x-y}alkyleneamino or H₂N—C_{x-y}alkyleneoxy.

Unlike alkyl, alkenyl consists of at least two carbon atoms, wherein at least two adjacent carbon atoms are joined together by a C—C double bond and a carbon atom can only be part of one C—C double bond. If in an alkyl as hereinbefore defined having at least two carbon atoms, two hydrogen atoms on adjacent carbon atoms are formally removed and the free valencies are saturated to form a second bond, the corresponding alkenyl is formed.

Examples of alkenyl are vinyl (ethenyl), prop-1-enyl, allyl (prop-2-enyl), isopropenyl, but-1-enyl, but-2-enyl, but-3-enyl, 2-methyl-prop-2-enyl, 2-methyl-prop-1-enyl, 1-methyl-prop-2-enyl, 1-methyl-prop-1-enyl, 1-methylidenepropyl, pent-1-enyl, pent-2-enyl, pent-3-enyl, pent-4-enyl, 3-methyl-but-3-enyl, 3-methyl-but-2-enyl, 3-methyl-but-1-enyl, hex-1-enyl, hex-2-enyl, hex-3-enyl, hex-4-enyl, hex-5-enyl, 2,3-dimethyl-but-3-enyl, 2,3-dimethyl-but-2-enyl, 2-methylidene-3-methylbutyl, 2,3-dimethyl-but-1-enyl, hexa-1,3-dienyl, hexa-1,4-dienyl, penta-1,4-dienyl, penta-1,3-dienyl, buta-1,3-dienyl, 2,3-dimethylbuta-1,3-diene etc.

By the generic terms propenyl, butenyl, pentenyl, hexenyl, butadienyl, pentadienyl, hexadienyl, heptadienyl, octadienyl, nonadienyl, decadienyl etc. without any further definition are meant all the conceivable isomeric forms with the corresponding number of carbon atoms, i.e. propenyl includes prop-1-enyl and prop-2-enyl, butenyl includes but-1-enyl, but-2-enyl, but-3-enyl, 1-methyl-prop-1-enyl, 1-methyl-prop-2-enyl etc.

Alkenyl may optionally be present in the cis or trans or E or Z orientation with regard to the double bond(s).

The above definition for alkenyl also applies when alkenyl is part of another (combined) group such as for example in C_{x-y}alkenylamino or C_{x-y}alkenyloxy.

Unlike alkylene, alkenylene consists of at least two carbon atoms, wherein at least two adjacent carbon atoms are joined together by a C—C double bond and a carbon atom can only be part of one C—C double bond. If in an alkylene as hereinbefore defined having at least two carbon atoms, two hydrogen atoms at adjacent carbon atoms are formally removed and the free valencies are saturated to form a second bond, the corresponding alkenylene is formed.

Examples of alkenylene are ethenylene, propenylene, 1-methylethenylene, butenylene, 1-methylpropenylene, 1,1-dimethylethenylene, 1,2-dimethylethenylene, pentenylene, 1,1-dimethylpropenylene, 2,2-dimethylpropenylene, 1,2-dimethylpropenylene, 1,3-dimethylpropenylene, hexenylene etc.

By the generic terms propenylene, butenylene, pentenylene, hexenylene etc. without any further definition are meant all the conceivable isomeric forms with the corresponding number of carbon atoms, i.e. propenylene includes 1-methylethenylene and butenylene includes 1-methylpropenylene, 2-methylpropenylene, 1,1-dimethylethenylene and 1,2-dimethylethenylene.

Alkenylene may optionally be present in the cis or trans or E or Z orientation with regard to the double bond(s).

The above definition for alkenylene also applies when alkenylene is a part of another (combined) group as for example in HO—C_{x-y}alkenyleneamino or H₂N—C_{x-y}alkenyleneoxy.

Unlike alkyl, alkynyl consists of at least two carbon atoms, wherein at least two adjacent carbon atoms are joined together by a C—C triple bond. If in an alkyl as hereinbefore defined having at least two carbon atoms, two hydrogen atoms in each case at adjacent carbon atoms are formally removed and the free valencies are saturated to form two further bonds, the corresponding alkynyl is formed.

Examples of alkynyl are ethynyl, prop-1-ynyl, prop-2-ynyl, but-1-ynyl, but-2-ynyl, but-3-ynyl, 1-methyl-prop-2-ynyl, pent-1-ynyl, pent-2-ynyl, pent-3-ynyl, pent-4-ynyl, 3-methyl-but-1-ynyl, hex-1-ynyl, hex-2-ynyl, hex-3-ynyl, hex-4-ynyl, hex-5-ynyl etc.

By the generic terms propynyl, butynyl, pentynyl, hexynyl, heptynyl, octynyl, nonynyl, decynyl etc. without any further definition are meant all the conceivable isomeric forms with the corresponding number of carbon atoms, i.e. propynyl includes prop-1-ynyl and prop-2-ynyl, butynyl includes but-1-ynyl, but-2-ynyl, but-3-ynyl, 1-methyl-prop-1-ynyl, 1-methyl-prop-2-ynyl, etc.

If a hydrocarbon chain carries both at least one double bond and also at least one triple bond, by definition it belongs to the alkynyl subgroup.

The above definition for alkynyl also applies if alkynyl is part of another (combined) group, as for example in C_{x-y}alkynylamino or C_{x-y}alkynyloxy.

Unlike alkylene, alkynylene consists of at least two carbon atoms, wherein at least two adjacent carbon atoms are joined together by a C—C triple bond. If in an alkylene as hereinbefore defined having at least two carbon atoms, two hydrogen atoms in each case at adjacent carbon atoms are formally removed and the free valencies are saturated to form two further bonds, the corresponding alkynylene is formed.

Examples of alkynylene are ethynylene, propynylene, 1-methylethynylene, butynylene, 1-methylpropynylene, 1,1-dimethylethynylene, 1,2-dimethylethynylene, pentynylene, 1,1-dimethylpropynylene, 2,2-dimethylpropynylene, 1,2-dimethylpropynylene, 1,3-dimethylpropynylene, hexynylene etc.

By the generic terms propynylene, butynylene, pentynylene, hexynylene etc. without any further definition are meant all the conceivable isomeric forms with the corresponding number of carbon atoms, i.e. propynylene includes 1-methylethynylene and butynylene includes 1-methylpropynylene, 2-methylpropynylene, 1,1-dimethylethynylene and 1,2-dimethylethynylene.

The above definition for alkynylene also applies if alkynylene is part of another (combined) group, as for example in HO—C_{x-y}alkynyleneamino or H₂N—C_{x-y}alkynyleneoxy.

By heteroatoms are meant oxygen, nitrogen and sulphur atoms.

Haloalkyl (haloalkenyl, haloalkynyl) is derived from the previously defined alkyl (alkenyl, alkynyl) by replacing one or more hydrogen atoms of the hydrocarbon chain independently of one another by halogen atoms, which may be identical or different. If a haloalkyl (haloalkenyl, haloalkynyl) is to be further substituted, the substitutions may take place independently of one another, in the form of mono- or polysubstitutions in each case, on all the hydrogen-carrying carbon atoms.

Examples of haloalkyl (haloalkenyl, haloalkynyl) are —CF₃, —CHF₂, —CH₂F, —CF₂CF₃, —CHFCF₃, —CH₂CF₃, —CF₂CH₃, —CHFCH₃, —CF₂CF₂CF₃, —CF₂CH₂CH₃, —CF═CF₂, —CCl═CH₂, —CBr═CH₂, —C≡C—CF₃, —CHFCH₂CH₃, —CHFCH₂CF₃ etc.

From the previously defined haloalkyl (haloalkenyl, haloalkynyl) are also derived the terms haloalkylene (haloalkenylene, haloalkenylene). Haloalkylene (haloalkenylene, haloalkynylene), unlike haloalkyl (haloalkenyl, haloalkynyl), is bivalent and requires two binding partners. Formally, the second valency is formed by removing a hydrogen atom from a haloalkyl (haloalkenyl, haloalkynyl).

Corresponding groups are for example —CH₂F and —CHF—, —CHFCH₂F and —CHFCHF— or >CFCH₂F etc.

The above definitions also apply if the corresponding halogen-containing groups are part of another (combined) group.

Halogen relates to fluorine, chlorine, bromine and/or iodine atoms.

Cycloalkyl is made up of the subgroups monocyclic hydrocarbon rings, bicyclic hydrocarbon rings and spiro-hydrocarbon rings. The systems are saturated. In bicyclic hydrocarbon rings two rings are joined together so that they have at least two carbon atoms together. In spiro-hydrocarbon rings one carbon atom (spiroatom) belongs to two rings together.

If a cycloalkyl is to be substituted, the substitutions may take place independently of one another, in the form of mono- or polysubstitutions in each case, on all the hydrogen-carrying carbon atoms. Cycloalkyl itself may be linked as a substituent to the molecule via every suitable position of the ring system.

Examples of cycloalkyl are cyclopropyl, cyclobutyl, cyclopentyl, cyclohexyl, cycloheptyl, bicyclo[2.2.0]hexyl, bicyclo[3.2.0]heptyl, bicyclo[3.2.1]octyl, bicyclo[2.2.2]octyl, bicyclo[4.3.0]nonyl (octahydroindenyl), bicyclo[4.4.0]decyl (decahydronaphthyl), bicyclo[2.2.1]heptyl (norbornyl), bicyclo[4.1.0]heptyl (norcaranyl), bicyclo[3.1.1]heptyl (pinanyl), spiro[2.5]octyl, spiro[3.3]heptyl etc.

The above definition for cycloalkyl also applies if cycloalkyl is part of another (combined) group as for example in C_{x-y}cycloalkylamino, C_{x-y}cycloalkyloxy or C_{x-y}cycloalkylalkyl.

If the free valency of a cycloalkyl is saturated, then an alicyclic group is obtained.

The term cycloalkylene can thus be derived from the previously defined cycloalkyl. Cycloalkylene, unlike cycloalkyl, is bivalent and requires two binding partners. Formally, the second valency is obtained by removing a hydrogen atom from a cycloalkyl. Corresponding groups are for example:
cyclohexyl and

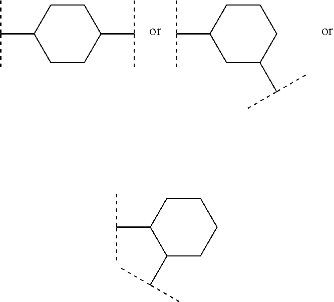

(cyclohexylene).

The above definition for cycloalkylene also applies if cycloalkylene is part of another (combined) group as for example in HO—C_{x-y}cycloalkyleneamino or H₂N—C_{x-y}cycloalkyleneoxy.

Cycloalkenyl is also made up of the subgroups monocyclic hydrocarbon rings, bicyclic hydrocarbon rings and spiro-hydrocarbon rings. However, the systems are unsaturated, i.e. there is at least one C—C double bond but no aromatic system. If in a cycloalkyl as hereinbefore defined two hydrogen atoms at adjacent cyclic carbon atoms are formally removed and the free valencies are saturated to form a second bond, the corresponding cycloalkenyl is obtained.

If a cycloalkenyl is to be substituted, the substitutions may take place independently of one another, in the form of mono- or polysubstitutions in each case, on all the hydrogen-carrying carbon atoms. Cycloalkenyl itself may be linked as a substituent to the molecule via every suitable position of the ring system.

Examples of cycloalkenyl are cycloprop-1-enyl, cycloprop-2-enyl, cyclobut-1-enyl, cyclobut-2-enyl, cyclopent-1-enyl, cyclopent-2-enyl, cyclopent-3-enyl, cyclohex-1-enyl, cyclohex-2-enyl, cyclohex-3-enyl, cyclohept-1-enyl, cyclohept-2-enyl, cyclohept-3-enyl, cyclohept-4-enyl, cyclobuta-1,3-dienyl, cyclopenta-1,4-dienyl, cyclopenta-1,3-dienyl, cyclopenta-2,4-dienyl, cyclohexa-1,3-dienyl, cyclohexa-1,5-dienyl, cyclohexa-2,4-dienyl, cyclohexa-1,4-dienyl, cyclohexa-2,5-dienyl, bicyclo[2.2.1]hepta-2,5-dienyl (norborna-2,5-dienyl), bicyclo[2.2.1]hept-2-enyl (norbornenyl), spiro[4,5]dec-2-enyl etc.

The above definition for cycloalkenyl also applies when cycloalkenyl is part of another (combined) group as for example in C_{x-y}cycloalkenylamino, C_{x-y}cycloalkenyloxy or C_{x-y}cycloalkenylalkyl.

If the free valency of a cycloalkenyl is saturated, then an unsaturated alicyclic group is obtained.

The term cycloalkenylene can thus be derived from the previously defined cycloalkenyl. Cycloalkenylene, unlike cycloalkenyl, is bivalent and requires two binding partners. Formally, the second valency is obtained by removing a hydrogen atom from a cycloalkenyl. Corresponding groups are for example:

cyclopentenyl and

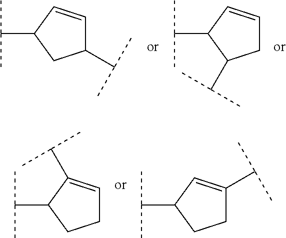

(cyclopentenylene) etc.

The above definition for cycloalkenylene also applies if cycloalkenylene is part of another (combined) group as for example in HO—C_{x-y}cycloalkenyleneamino or H₂N—C_{x-y}cycloalkenyleneoxy.

Aryl denotes mono-, bi- or tricyclic carbocycles with at least one aromatic carbocycle. Preferably, it denotes a monocyclic group with six carbon atoms (phenyl) or a bicyclic group with nine or ten carbon atoms (two six-membered rings or one six-membered ring with a five-membered ring), wherein the second ring may also be aromatic or, however, may also be saturated or partially saturated.

If an aryl is to be substituted, the substitutions may take place independently of one another, in the form of mono- or polysubstitutions in each case, on all the hydrogen-carrying carbon atoms. Aryl itself may be linked as a substituent to the molecule via every suitable position of the ring system.

Examples of aryl are phenyl, naphthyl, indanyl (2,3-dihydroindenyl), indenyl, anthracenyl, phenanthrenyl, tetrahydronaphthyl (1,2,3,4-tetrahydronaphthyl, tetralinyl), dihydronaphthyl (1,2-dihydronaphthyl), fluorenyl etc.

The above definition of aryl also applies if aryl is part of another (combined) group as for example in arylamino, aryloxy or arylalkyl.

If the free valency of an aryl is saturated, then an aromatic group is obtained.

The term arylene can also be derived from the previously defined aryl. Arylene, unlike aryl, is bivalent and requires two binding partners. Formally, the second valency is formed by removing a hydrogen atom from an aryl. Corresponding groups are for example:
phenyl and

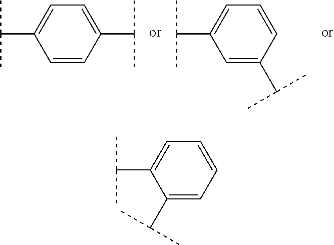

(o, m, p-phenylene),
naphthyl and

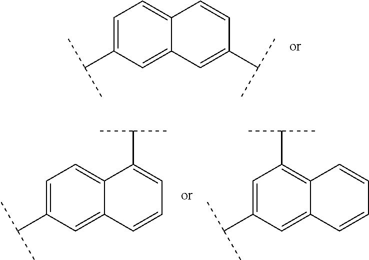

etc.

The above definition for arylene also applies if arylene is part of another (combined) group as for example in HO-aryleneamino or H₂N-aryleneoxy.

Heterocyclyl denotes ring systems, which are derived from the previously defined cycloalkyl, cycloalkenyl and aryl by replacing one or more of the groups —CH₂-independently of one another in the hydrocarbon rings by the groups —O—, —S— or —NH— or by replacing one or more of the groups ═CH— by the group ═N—, wherein a total of not more than five heteroatoms may be present, at least one carbon atom may be present between two oxygen atoms and between two sulphur atoms or between one oxygen and to one sulphur atom and the ring as a whole must have chemical stability. Heteroatoms may optionally be present in all the possible oxidation stages (sulphur→sulphoxide —SO—, sulphone —SO₂—; nitrogen→N-oxide). In a heterocyclyl there is no heteroaromatic ring, i.e. no heteroatom is part of an aromatic system.

A direct result of the derivation from cycloalkyl, cycloalkenyl and aryl is that heterocyclyl is made up of the subgroups monocyclic heterorings, bicyclic heterorings, tricyclic heterorings and spiro-heterorings, which may be present in saturated or unsaturated form.

By unsaturated is meant that there is at least one double bond in the ring system in question, but no heteroaromatic system is formed. In bicyclic heterorings two rings are linked together so that they have at least two (hetero)atoms in common. In spiro-heterorings one carbon atom (spiroatom) belongs to two rings together.

If a heterocyclyl is substituted, the substitutions may take place independently of one another, in the form of mono- or polysubstitutions in each case, on all the hydrogen-carrying carbon and/or nitrogen atoms. Heterocyclyl itself may be linked as a substituent to the molecule via every suitable position of the ring system.

Examples of heterocyclyl are tetrahydrofuryl, pyrrolidinyl, pyrrolinyl, imidazolidinyl, thiazolidinyl, imidazolinyl, pyrazolidinyl, pyrazolinyl, piperidinyl, piperazinyl, oxiranyl, aziridinyl, azetidinyl, 1,4-dioxanyl, azepanyl, diazepanyl, morpholinyl, thiomorpholinyl, homomorpholinyl, homopiperidinyl, homopiperazinyl, homothiomorpholinyl, thiomorpholinyl-S-oxide, thiomorpholinyl-S,S-dioxide, 1,3-dioxolanyl, tetrahydropyranyl, tetrahydrothiopyranyl, [1,4]-oxazepanyl, tetrahydrothienyl, homothiomorpholinyl-S,S-dioxide, oxazolidinonyl, dihydropyrazolyl, dihydropyrrolyl, dihydropyrazinyl, dihydropyridyl, dihydro-pyrimidinyl, dihydrofuryl, dihydropyranyl, tetrahydrothienyl-S-oxide, tetrahydrothienyl-S,S-dioxide, homothiomorpholinyl-S-oxide, 2,3-dihydroazet, 2H-pyrrolyl, 4H-pyranyl, 1,4-dihydropyridinyl, 8-aza-bicyclo[3.2.1]octyl, 8-aza-bicyclo[5.1.0]octyl, 2-oxa-5-azabicyclo[2.2.1]heptyl, 8-oxa-3-aza-bicyclo[3.2.1]octyl, 3,8-diaza-bicyclo[3.2.1]octyl, 2,5-diaza-bicyclo[2.2.1]heptyl, 1-aza-bicyclo[2.2.2]octyl, 3,8-diaza-bicyclo[3.2.1]octyl, 3,9-diaza-bicyclo[4.2.1]nonyl, 2,6-diaza-bicyclo[3.2.2]nonyl, 1,4-dioxa-spiro[4.5]decyl, 1-oxa-3,8-diaza-spiro[4.5]decyl, 2,6-diaza-spiro[3.3]heptyl, 2,7-diaza-spiro[4.4]nonyl, 2,6-diaza-spiro[3.4]octyl, 3,9-diaza-spiro[5.5]undecyl, 2.8-diaza-spiro[4,5]decyl etc.

Further examples are the structures illustrated below, which may be attached via each hydrogen-carrying atom (exchanged for hydrogen):

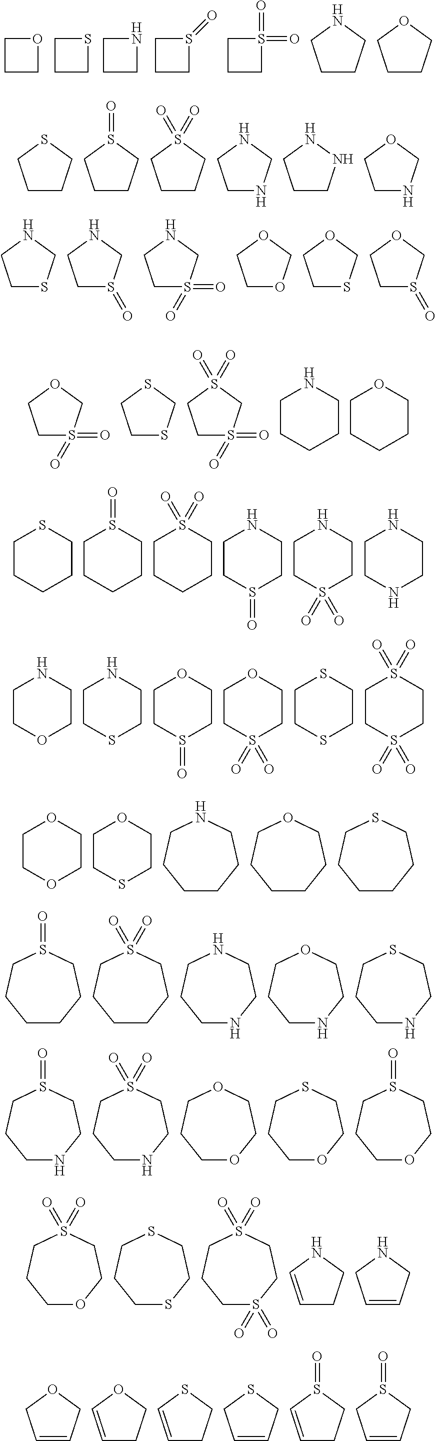

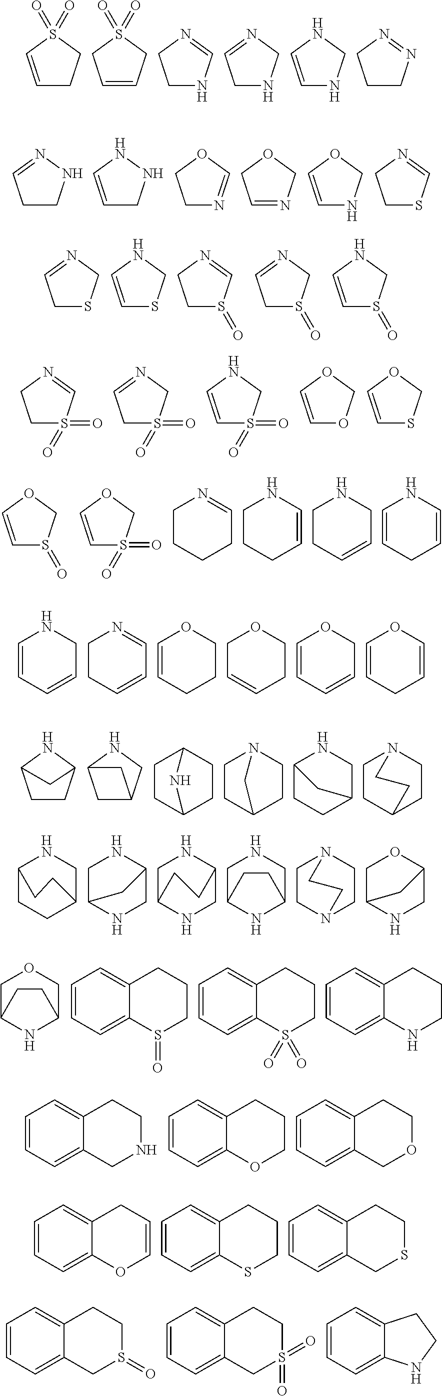

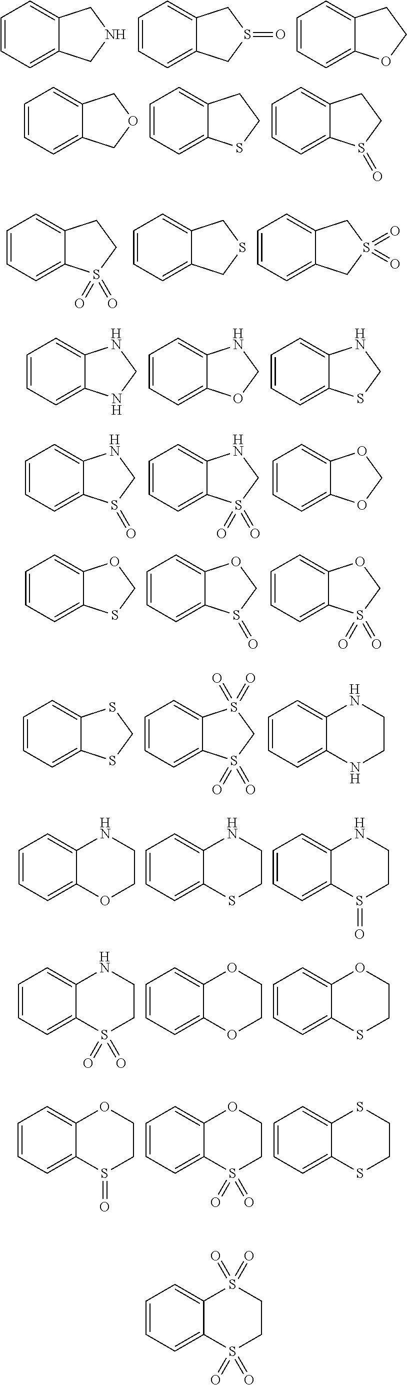

The above definition of heterocyclyl also applies if heterocyclyl is part of another (combined) group as for example in heterocyclylamino, heterocyclyloxy or heterocyclylalkyl.

If the free valency of a heterocyclyl is saturated, then a heterocyclic group is obtained.

The term heterocyclylene is also derived from the previously defined heterocyclyl. Heterocyclylene, unlike heterocyclyl, is bivalent and requires two binding partners. Formally, the second valency is obtained by removing a hydrogen atom from a heterocyclyl. Corresponding groups are for example:
piperidinyl and

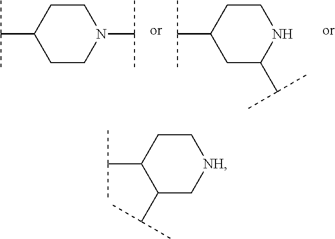

2,3-dihydro-1H-pyrrolyl and

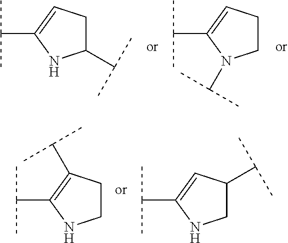

etc.

The above definition of heterocyclylene also applies if heterocyclylene is part of another (combined) group as for example in HO-heterocyclyleneamino or H₂N-heterocyclyleneoxy.

Heteroaryl denotes monocyclic heteroaromatic rings or polycyclic rings with at least one heteroaromatic ring, which compared with the corresponding aryl or cycloalkyl (cycloalkenyl) contain, instead of one or more carbon atoms, one or more identical or different heteroatoms, selected independently of one another from among nitrogen, sulphur and oxygen, wherein the resulting group must be chemically stable. The prerequisite for the presence of heteroaryl is a heteroatom and a heteroaromatic system.

If a heteroaryl is to be substituted, the substitutions may take place independently of one another, in the form of mono- or polysubstitutions in each case, on all the hydrogen-carrying carbon and/or nitrogen atoms. Heteroaryl itself may be linked as a substituent to the molecule via every suitable position of the ring system, both carbon and nitrogen.

Examples of heteroaryl are furyl, thienyl, pyrrolyl, oxazolyl, thiazolyl, isoxazolyl, isothiazolyl, pyrazolyl, imidazolyl, triazolyl, tetrazolyl, oxadiazolyl, thiadiazolyl, pyridyl, pyrimidyl, pyridazinyl, pyrazinyl, triazinyl, pyridyl-N-oxide, pyrrolyl-N-oxide, pyrimidinyl-N-oxide, pyridazinyl-N-oxide, pyrazinyl-N-oxide, imidazolyl-N-oxide, isoxazolyl-N-oxide, oxazolyl-N-oxide, thiazolyl-N-oxide, oxadiazolyl-N-oxide, thiadiazolyl-N-oxide, triazolyl-N-oxide, tetrazolyl-N-oxide, indolyl, isoindolyl, benzofuryl, benzothienyl, benzoxazolyl, benzothiazolyl, benzisoxazolyl, benzisothiazolyl, benzimidazolyl, indazolyl, isoquinolinyl, quinolinyl, quinoxalinyl, cinnolinyl, phthalazinyl, quinazolinyl, benzotriazinyl, indolizinyl, oxazolopyridyl, imidazopyridyl, naphthyridinyl, benzoxazolyl, pyridopyridyl, pyrimidopyridyl, purinyl, pteridinyl, benzothiazolyl, imidazopyridyl, imidazothiazolyl, quinolinyl-N-oxide, indolyl-N-oxide, isoquinolyl-N-oxide, quinazolinyl-N-oxide, quinoxalinyl-N-oxide, phthalazinyl-N-oxide, indolizinyl-N-oxide, indazolyl-N-oxide, benzothiazolyl-N-oxide, benzimidazolyl-N-oxide etc.

Further examples are the structures illustrated below, which may be attached via each hydrogen-carrying atom (exchanged for hydrogen):

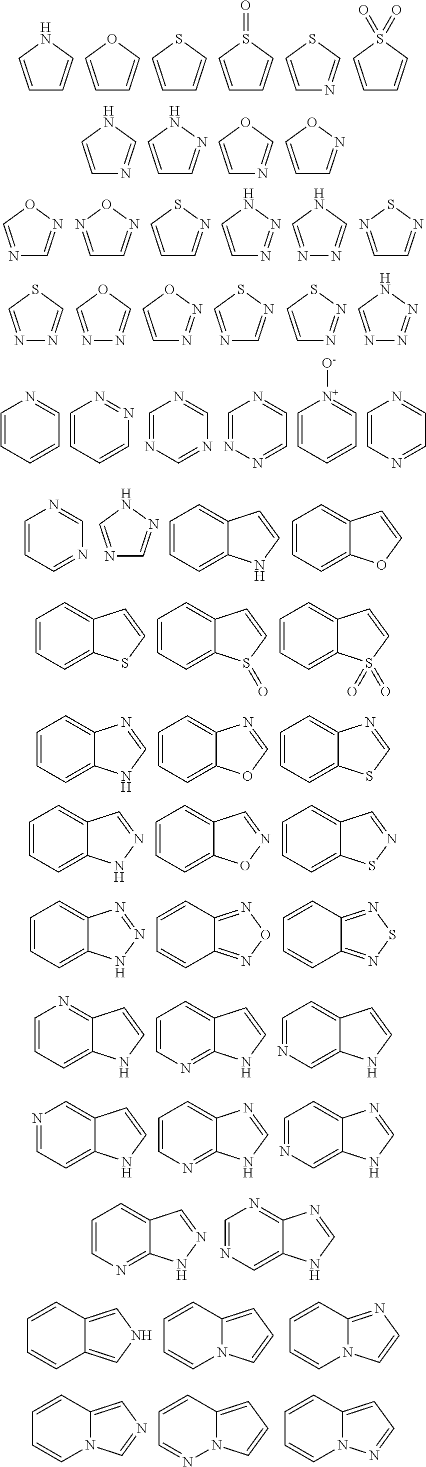

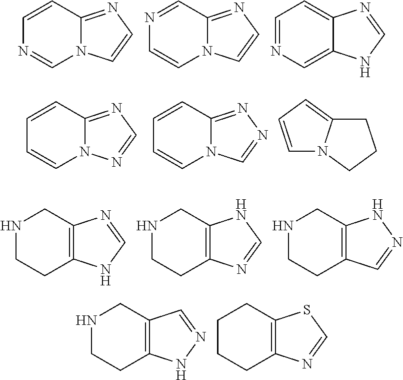

The above definition of heteroaryl also applies if heteroaryl is part of another (combined) group as for example in heteroarylamino, heteroaryloxy or heteroarylalkyl.

If the free valency of a heteroaryl is saturated, a heteroaromatic group is obtained.

The term heteroarylene is also derived from the previously defined heteroaryl. Heteroarylene, unlike heteroaryl, is bivalent and requires two binding partners. Formally, the second valency is obtained by removing a hydrogen atom from a heteroaryl. Corresponding groups are for example:
pyrrolyl and

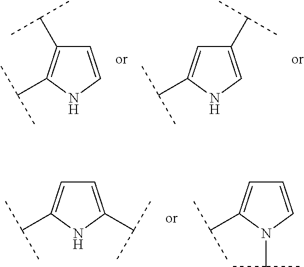

etc.

The above definition of heteroarylene also applies if heteroarylene is part of another (combined) group as for example in HO-heteroaryleneamino or H₂N-heteroaryleneoxy.

By substituted is meant that a hydrogen atom which is bound directly to the atom under consideration, is replaced by another atom or another group of atoms (substituent). Depending on the starting conditions (number of hydrogen atoms) mono- or polysubstitution may take place on one atom. Substitution with a particular substituent is only possible if the permitted valencies of the substituent and of the atom that is to be substituted correspond to one another and the substitution leads to a stable compound (i.e. to a compound which is not converted spontaneously, e.g. by rearrangement, cyclisation or elimination).

Bivalent substituents such as ═S, ═NR, ═NOR, ═NNRR, ═NN(R)C(O)NRR, ═N₂ or the like, may only be substituents at carbon atoms, wherein the bivalent substituent ═O may also be a substituent at sulphur. Generally, substitution may be carried out by a bivalent substituent only at ring systems and requires replacement by two geminal hydrogen atoms, i.e. hydrogen atoms that are bound to the same carbon atom that is saturated prior to the substitution. Substitution by a bivalent substituent is therefore only possible at the group —CH₂— or sulphur atoms of a ring system.

Stereochemistry/solvates/hydrates: Unless specifically indicated, throughout the specification and appended claims, a given chemical formula or name shall encompass tautomers and all stereo, optical and geometrical isomers (e.g. enantiomers, diastereomers, E/Z isomers, etc.) and racemates thereof as well as mixtures in different proportions of the separate enantiomers, mixtures of diastereomers, or mixtures of any of the foregoing forms where such isomers and enantiomers exist, as well as salts, including pharmaceutically acceptable salts thereof. The compounds and salts of the invention can exist in unsolvated as well as solvated forms with pharmaceutically acceptable solvents such as water, ethanol and the like. In general, the solvated forms such as hydrates are considered equivalent to the unsolvated forms for the purposes of the invention.

Salts: The phrase “pharmaceutically acceptable” is employed herein to refer to those compounds, materials, compositions, and/or dosage forms which are, within the scope of sound medical judgement, suitable for use in contact with the tissues of human beings and animals without excessive toxicity, irritation, allergic response, or other problem or complication, and commensurate with a reasonable benefit/risk ratio.

As used herein “pharmaceutically acceptable salts” refers to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like.

For example, such salts include acetates, ascorbates, benzenesulphonates, benzoates, besylates, bicarbonates, bitartrates, bromides/hydrobromides, Ca-edetates/edetates, camsylates, carbonates, chlorides/hydrochlorides, citrates, edisylates, ethane disulphonates, estolates esylates, fumarates, gluceptates, gluconates, glutamates, glycolates, glycollylarsnilates, hexylresorcinates, hydrabamines, hydroxymaleates, hydroxynaphthoates, iodides, isothionates, lactates, lactobionates, malates, maleates, mandelates, methanesulphonates, mesylates, methylbromides, methylnitrates, methylsulphates, mucates, napsylates, nitrates, oxalates, pamoates, pantothenates, phenyl acetates, phosphates/diphosphates, polygalacturonates, propionates, salicylates, stearates, subacetates, succinates, sulphamides, sulphates, tannates, tartrates, teoclates, toluenesulphonates, triethiodides, ammonium, benzathines, chloroprocaines, cholines, diethanolamines, ethylenediamines, meglumines and procaines.

Further pharmaceutically acceptable salts can be formed with cations from metals like aluminium, calcium, lithium, magnesium, potassium, sodium, zinc and the like (also see Pharmaceutical salts, Birge, S. M. et al., J. Pharm. Sci., (1977), 66, 1-19).

The pharmaceutically acceptable salts of the present invention can be synthesised from the parent compound which contains a basic or acidic moiety by conventional chemical methods. Generally, such salts can be prepared by reacting the free acid or base form of these compounds with a sufficient amount of the appropriate base or acid in water or in an organic diluent like ether, ethyl acetate, ethanol, isopropanol, or acetonitrile, or a mixture thereof.

Salts of other acids than those mentioned above which for example are useful for purifying or isolating the compounds of the present invention (e.g. trifluoroacetates), also comprise a part of the invention.

Some abbreviated notations and their structure correspondences are listed below:

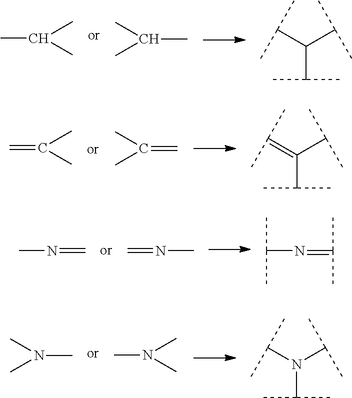

If for example in the sequence X-Y-Z the component Y is supposed to correspond to the structural section —N═, this means both X═N—Z and also X—N═Z.

In a representation such as for example

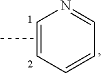

the dotted line means that the ring system may be attached to the molecule via the carbon atom 1 or 2, and is thus equivalent to the following representation

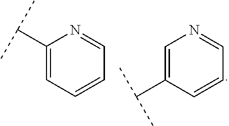

In a representation such as for example

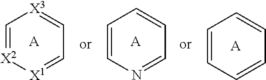

the letter A has the function of a ring designation in order to make it easier, for example, to indicate the attachment of the ring in question to other rings.

For bivalent groups in which it is crucial to determine which adjacent groups they bind and with which valency, the corresponding binding partners are indicated in brackets where necessary for clarification purposes, as in the following representations:

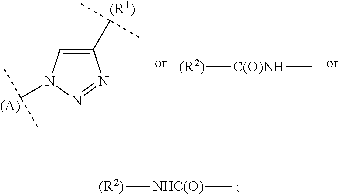

Groups or substituents are frequently selected from among a number of alternative groups/substituents with a corresponding group designation (e.g. R^{a}, R^{b} etc). If such a group is used repeatedly to define a compound according to the invention in different parts of the molecule, it must always be borne in mind that the various uses are to be regarded as totally independent of one another.

By a therapeutically effective amount for the purposes of this invention is meant a quantity of substance that is capable of obviating symptoms of illness or of preventing or alleviating these symptoms, or which prolong the survival of a treated patient.

List of abbreviations

| | |
|---|---|
| Ac | acetyl |
| aq. | aquatic, aqueous |
| ATP | adenosine triphosphate |
| BiPh | biphenyl |
| Bn | benzyl |
| Boc | tert-butyloxycarbonyl |
| Bu | butyl |
| c | concentration |
| d | day(s) |
| dba | dibenzylideneacetone |
| TLC | thin layer chromatography |
| DCM | dichloromethane |
| DEA | diethylamine |
| DIPEA | N-ethyl-N,N-diisopropylamine (Hunig's base) |
| DMAP | 4-N,N-dimethylaminopyridine |
| DME | 1,2-dimethoxyethane |
| DMF | N,N-dimethylformamide |
| DMSO | dimethylsulphoxide |
| DPPA | diphenylphosphorylazide |
| dppf | 1.1′-bis(diphenylphosphino)ferrocene |
| EDTA | ethylenediaminetetraacetic acid |
| EGTA | ethyleneglycoltetraacetic acid |
| eq | equivalent(s) |
| ESI | electron spray ionization |
| Et | ethyl |
| Et₂O | diethyl ether |
| EtOAc | ethyl acetate |
| EtOH | ethanol |
| h | hour |
| HATU | O-(7-azabenzotriazol-1-yl)-N,N,N′,N′-tetramethyl- |
| | uronium hexafluorophosphate |
| HPLC | high performance liquid chromatography |
| i | iso |
| Kat., kat. | catalyst, catalytic |
| conc. | concentrated |
| LC | liquid chromatography |
| sln. | solution |
| Me | methyl |
| MeOH | methanol |
| min | minutes |
| MPLC | medium pressure liquid chromatography |
| MS | mass spectrometry |
| NBS | N-Bromo-succinimide |
| NMM | N-methylmorpholine |
| NMP | N-methylpyrrolidone |
| NP | normal phase |
| n.a. | not available |
| PBS | phosphate-buffered saline |
| Ph | phenyl |
| Pr | propyl |
| Py | pyridine |
| rac | racemic |
| red. | reduction |
| R_{f} (Rf) | retention factor |
| RP | reversed phase |
| rt | ambient temperature |
| S_{N} | nucleophilic substitution |
| TBAF | tetrabutylammonium fluoride |
| TBDMS | tert-butyldimethylsilyl |
| TBME | tert-butylmethylether |
| TBTU | O-(benzotriazol-1-yl)-N,N,N′,N′-tetramethyl-uronium |
| | tetrafluoroborate |
| tBu | tert-butyl |
| TEA | triethylamine |
| temp. | temperature |
| tert | tertiary |
| Tf | triflate |
| TFA | trifluoroacetic acid |
| THF | tetrahydrofuran |
| TMS | trimethylsilyl |
| t_{Ret.} | retention time (HPLC) |
| TRIS | tris(hydroxymethyl)-aminomethane |
| TsOH | p-toluenesulphonic acid |
| UV | ultraviolet |

Features and advantages of the present invention will become apparent from the following detailed examples which illustrate the fundamentals of the invention by way of example without restricting its scope:

### Preparation of the Compounds According to the Invention

### General

Unless stated otherwise, all the reactions are carried out in commercially obtainable apparatus using methods that are commonly used in chemical laboratories. Starting materials that are sensitive to air and/or moisture are stored under protective gas and corresponding reactions and manipulations therewith are carried out under protective gas (nitrogen or argon).

The compounds according to the invention are named in accordance with CAS rules using the software Autonom (Beilstein).

Microwave reactions are carried out in an initiator/reactor made by Biotage or in an Explorer made by CEM in sealed containers (preferably 2, 5 or 20 mL), preferably with stirring.

### Chromatography

For preparative medium pressure chromatography (MPLC) silica gel made by Millipore (name: Granula Silica Si-60A 35-70 μm, NP phase) or C-18 RP-silica gel (RP-phase) made by Macherey Nagel (name: Polygoprep 100-50 C18) is used. Automated normal phase chromatography is also carried out on a CombiFlash Companion XL apparatus in combination with a CombiFlash Foxy 200 fraction collector made by Isco. For this, commercially obtainable RediSepRf (120 g silica gel) one-way columns are used. Furthermore, automated normal phase chromatography can also be carried out on an Isolera Flash Purification apparatus made by Biotage. For this, commercially obtainable one-way SNAP-Cartridges (e.g. 50 g silica gel) are used.

The thin layer chromatography is carried out on ready-made silica gel 60 TLC plates on glass (with fluorescence indicator F-254) made by Merck.

The preparative high pressure chromatography (RP HPLC) of the example compounds according to the invention is carried out with columns made by Waters (names: XTerra Prep. MS C18, 5 μm, 30×100 mm or XTerra Prep. MS C18, 5 μm, 50×100 mm OBD or Symmetrie C18, 5 μm, 19×100 mm or Sunfire C18 OBD, 19×100 mm, 5 μm or Sunfire Prep C10 μm OBD 50×150 mm or X-Bridge Prep C18 5 μm OBD 19×50 mm) or X-Bridge Prep C18 10 μm OBD 50×150 mm), Agilent (name: Zorbax SB-C8 5 μm PrepHT 21.2×50 mm) and Phenomenex (names: Gemini C18 5 μm AXIA 21.2×50 mm or Gemini C18 10 μm 50×150 mm). Different gradients of H₂O/acetonitrile or H₂O/MeOH are used to elute the compounds, while 0.1% HCOOH is added to the water (acidic conditions). For the chromatography under basic conditions H₂O/acetonitrile gradients are used as well, while the water is made alkaline as follows: 5 mL NH₄HCO₃ solution (158 g in 1 L H₂O) and 2 mL NH₃ (7 M in MeOH) are replenished to 1 L with H₂O.

The preparative high pressure chromatography on normal phase (NP HPLC) of the example compounds according to the invention is carried out with columns made by Macherey & Nagel (name: Nucleosil, 50-7, 40×250 mm) and VDSoptilab (name: Kromasil 100 NH₂, 10 μM, 50×250 mm). Different gradients of DCM/MeOH are used to to elute the compounds, while 0.1% NH₃ is added to the MeOH.

The analytical HPLC (reaction control) of intermediate compounds is carried out using columns made by Agilent (names: Zorbax SB-C8, 5 μm, 21.2×50 mm or Zorbax SB-C₈-3.5 μm 2.1×50 mm) and Phenomenex (name: Gemini C18 3 μm 2×30 mm). The analytical equipment is also equipped with a mass detector in each case.

### HPLC-Mass Spectroscopy/UV-Spectrometry

The retention times/MS-ESL⁺ for characterizing the example compounds according to the invention are produced using an HPLC-MS apparatus (high performance liquid chromatography with mass detector). Compounds that elute at the injection peak are given the retention time t_{Ret.}=0.00.

### HPLC-MS Method A

HPLC: Agilent 1100 Series
MS: Agilent LC/MSD SL
Column: Waters, XBridge™ C18, 2.5 μm, 2.1×20 mm (Part. No. 186003201)
Eluent: A: 0.1% NH₃ (=pH 9-10)
   B: Acetonitrile HPLC grade
Detection: MS: Positive and negative mode
Mass Range: 120-800 m/z
Flow: 1.00 mL/min
Column temperature: 60° C.
Gradient: 0.00 min 5% B
   0.00-2.50 min 5%→95% B
   2.51-2.80 min 95% B
   2.81-3.10 min 95%→5% B

The compounds according to the invention are prepared by the methods of synthesis described hereinafter in which the substituents of the general formulae have the meanings given hereinbefore. These methods are intended as an illustration of the invention without restricting its subject matter and the scope of the compounds claimed to these examples. Where the preparation of starting compounds is not described, they are commercially obtainable or may be prepared analogously to known compounds or methods described herein. Substances described in the literature are prepared according to the published methods of synthesis.

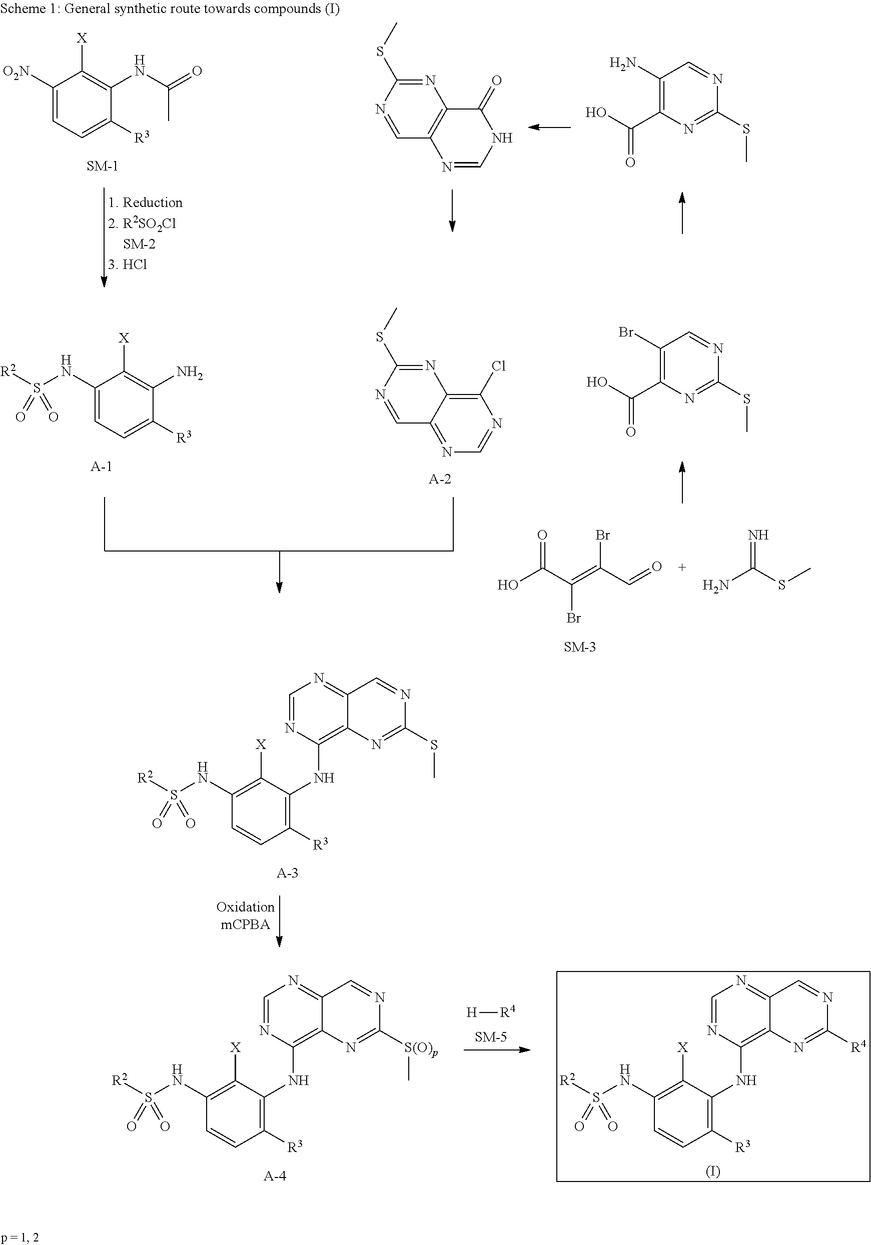

Compounds (I) according to the invention can be prepared as depicted in the general reaction scheme 1 starting from starting materials SM-1 to SM-4 which are either commercially available or can be synthesized as described below.

Compounds (I) are synthesized via a nucleophilic substitution reaction of the sulfinyl- or sulfanyl-derivative A-4 with nucleophiles H—R⁴ (SM-5), e.g. 1° and 2° amines, nitrogen-containing heteroaryls etc.

The sulfinyl- or sulfanyl-derivatives A-4 are activated forms for a nucleophilic substitution which are obtained from the methylthio-derivatives A-3 via oxidation with common reagents like e.g. 3-chloro-peroxybenzoic acid (mCPBA), sodium metaperiodate, hydrogen peroxide etc. Alternatively, starting materials bearing other well-known leaving groups like e.g. chlorine, fluorine, iodine etc. could be used.

Methylthio-derivatives A-3 are synthesized from building blocks A-1 and A-2 via a nucleophilic substitution reaction:

Anilines A-1 are obtained from nitro compounds SM-1 via reduction of the nitro function, to sulfone amide formation with sulfonic acid chlorides or sulfamoyl chlorides and subsequent deprotection of the amino function with e.g. aqueous HCl.

8-Chloro-2-(methylthio)pyrimido[5,4-d]pyrimidine A-2 is commercially available or can be readily prepared according to published procedures, e.g. as described in WO 1997/32882 or WO 1996/07657.

Compounds (I) which are directly synthesized following the synthetic route depicted in scheme 1 and which carry functional groups, either in R² or R⁴, that can be further modified such as e.g. halogen atoms, amino and hydroxy groups (including cyclic amines), carboxylic acid or ester functions, nitrils etc. can be optionally derivatized to further compounds (I) by well established organic chemical transformations such as palladium-catalyzed cross coupling reactions, acylation, amidation, addition, reduction or (reductive) alkylation. These additional steps are not depicted in scheme 1.

Likewise, it is also possible to include these additional steps in the synthetic routes depicted in scheme 1, i.e. to carry out derivatization reactions with intermediate compounds.

In addition, it may also be possible that building blocks bearing protecting groups are used, i.e. further steps for deprotection are necessary.

### A. Synthesis of Starting Materials

### A.1. Synthesis of SM-1

### A.1.1. Experimental Procedure for the Synthesis of SM-1a

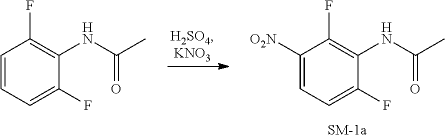

2,6-Difluoroacetanilide (4.00 g, 23.4 mmol) is taken-up in concentrated H₂SO₄ (10 mL) and cooled to −10° C. KNO₃ (4.73 g, 46.7 mmol) is added in small portions with stirring. After complete addition the cooling bath is removed, the mixture is slowly warmed to rt and stirred for 2 h. The reaction mixture is poured into ice water. The resulting precipitate is filtered off and dried in vacuo to give SM-1a (HPLC-MS: t_{Ret}. =0.50 min; MS (M+H)⁺=215) which is used without further purification.

B. Synthesis of Compounds (I) Starting from SM-1

### B.1. Synthesis of Anilines A-1

### B.1.1 Experimental Procedure for the Synthesis of Aniline A-1a

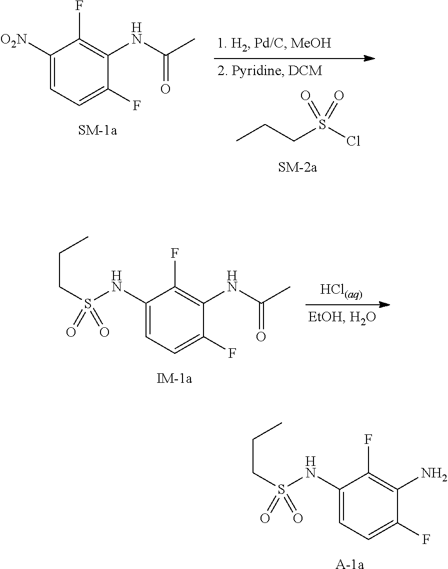

SM-1a (55.0 g, 254 mmol) is taken-up in MeOH (1.0 L). Pd/C (10.0 g, 10%) is added and the mixture is hydrogenated in an autoclave at 200 psi for 3 h. The reaction mixture is filtered and concentrated under reduced pressure. The residue is purified by column chromatography on silica gel using DCM/MeOH (96:4) as eluent. The product containing fractions of the aniline intermediate (HPLC-MS: t_{Ret.}=0.25 min; MS (M−H)⁻=185) are combined and evaporated. To the aniline intermediate (35.0 g, 188 mmol) in DCM (100 mL) and pyridine (6.6 mL, 75 mmol) is added n-propane sulfonyl chloride SM-2a (29.5 mL, 263 mmol) and the mixture is stirred at rt for 16 h. The reaction mixture is diluted with EtOAc (200 mL) and the layers are separated. The organic layer is washed with water and hydrochloric acid (1 N), dried over MgSO₄ and evaporated to yield IM-1a which was used without further purification.

IM-1a (38.0 g, 130 mmol) is taken-up in EtOH (250 mL), H₂O (200 mL) and concentrated hydrochloric acid (200 mL) and heated to 80° C. for 2 h. The reaction mixture is concentrated under reduced pressure, aqueous NaOH (4 N) is added until pH=6 is reached and the mixture is extracted 2× with DCM. The combined organic layer is washed with saturated NaCl solution, dried over MgSO₄, filtered and evaporated to give A-1a (HPLC-MS: t_{Ret.}=0.22 min; MS (M−H⁻=249) as a hydrochloride which was used without further purification.

Analogously to this procedure additional anilines A-1 can be prepared (also using other nitro compounds SM-1) with various sulfonyl chlorides SM-2.

TABLE 1
Anilines A-1

| | | t_{Ret.} | MS |
|---|---|---|---|
| | | (HPLC) | (M + |
| # | structure | [min] | H)⁺ |
| A-1a | 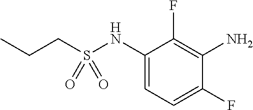 [SMILES: CCCS(=O)(=O)Nc1ccc(F)c(N)c1F] | 1.38 | 251 |
| A-1b | 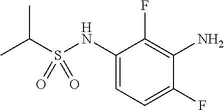 [SMILES: CC(C)S(=O)(=O)Nc1ccc(F)c(N)c1F] | n.d. | 251 |
| A-1c | 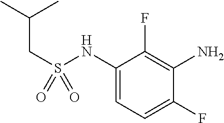 [SMILES: CC(C)CS(=O)(=O)Nc1ccc(F)c(N)c1F] | n.d. | 265 |
| A-1d | 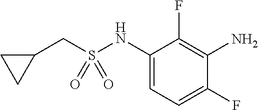 [SMILES: Nc1c(F)ccc(NS(=O)(=O)CC2CC2)c1F] | n.d. | 263 |
| A-1e | 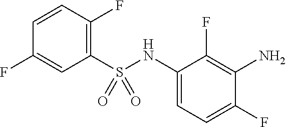 [SMILES: Nc1c(F)ccc(NS(=O)(=O)c2cc(F)ccc2F)c1F] | n.d. | 321 |

### B.2. Synthesis of Compounds A-3

### B.2.1 Experimental Procedure for the Synthesis of Compound A-3a

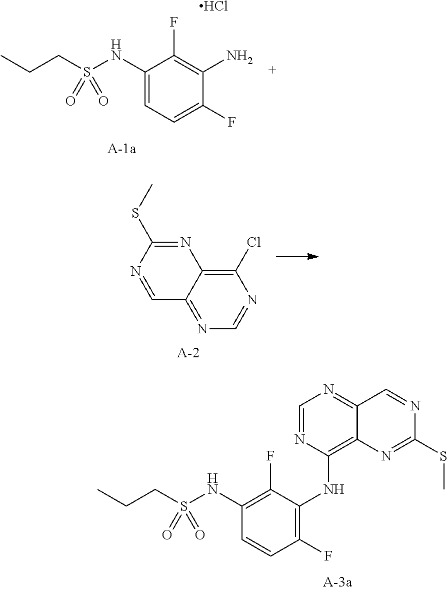

A-1a (4.0 g, 13.951 mmol) and A-2 (2.967 g, 13.952 mmol) are dissolved in acetic acid (25 mL). The reaction mixture is stirred for 2 h at 50° C. and concentrated in vacuo. Some MeOH is added to dissolve solid particles. The reaction mixture is then poured on crushed ice (200 mL). The obtained solid is filtered off and dried in vacuo to give the crude product A-3a (HPLC-MS: t_{Ret.}=0.85 min; MS (M+H)⁺=427) which is used without further purification.

Analogously to this procedure additional sulfonamides A-3 can be prepared using different anilines A-1.

### B.3. Synthesis of Compounds A-4

### B.3.1 Experimental Procedure for the Synthesis of Compound A-4-a

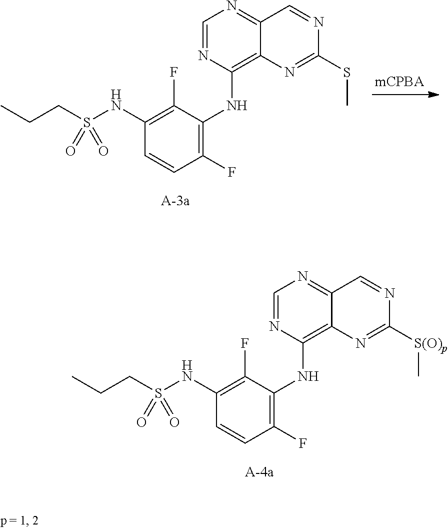

A-3a (5.9 g, 13.835 mmol) is suspended in DCM (50 mL). mCPBA (3.455 g, 15.416 mmol) is added and the reaction mixture is stirred for 30 min at rt. The organic phase is washed 2× with saturated NaHCO₃ solution, dried over MgSO₄ and concentrated in vacuo to give the crude product A-4-a (mixture of sulfon and sulfoxid, HPLC-MS: t_{Ret.}=0 min; MS (M+H)⁺=443/459) which is used without further purification.

### B.4. Synthesis of Final Compounds (I)

### B.4.1. Experimental Procedure for the Synthesis of Example 1-1

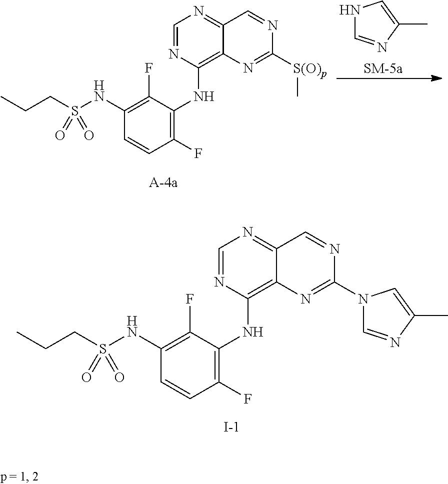

4-Methylimidazole SM-5a (37.11 mg, 0.452 mmol) and DIPEA (200 μL, 1.176 mmol) are added into a vial. The sulfoxide/sulfone A-4-a (100 mg, 0.226 mmol) is dissolved in NMP (900 μL) and subsequently added to the vial. The reaction mixture is shaken for 16 h at 50° C., filtered and purified by basic RP HPLC. The product containing fractions of I-1 (HPLC-MS: t_{Ret.}=0.88 min; MS (M+H)⁺=480) are freeze dried.

Analogously to procedure B.4.1. (synthesis of I-1) additional compounds I-2 to I-36 are to prepared using various amines and nitrogen-containing heteroaryls (SM-5).

TABLE 2
Structures and analytical data of example compounds I-1 to I-36.

| | | t_{Ret.} | |
|---|---|---|---|
| | | (HPLC) | MS |
| # | Structure | [min] | (M + H)⁺ |
| I-1 | 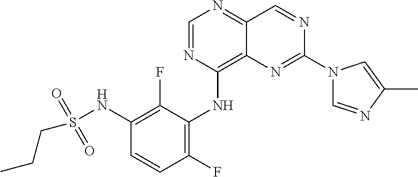 [SMILES: CCCS(=O)(=O)Nc1ccc(F)c(Nc2ncnc3cnc(-n4cnc(C)c4)nc23)c1F] | 0.83 | 461 |
| I-2 | 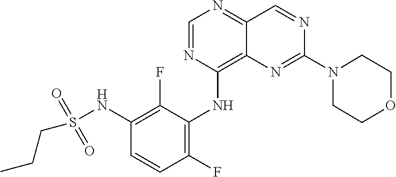 [SMILES: CCCS(=O)(=O)Nc1ccc(F)c(Nc2ncnc3cnc(N4CCOCC4)nc23)c1F] | 1.12 | 466 |
| I-3 | 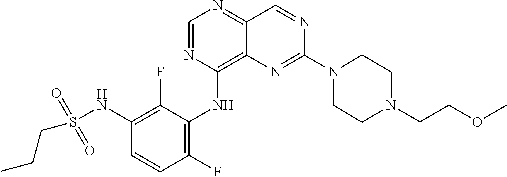 [SMILES: CCCS(=O)(=O)Nc1ccc(F)c(Nc2ncnc3cnc(N4CCN(CCOC)CC4)nc23)c1F] | 1.13 | 523 |
| I-4 | 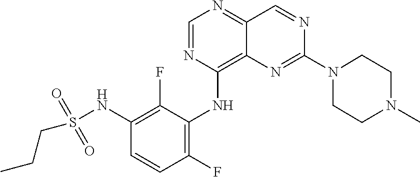 [SMILES: CCCS(=O)(=O)Nc1ccc(F)c(Nc2ncnc3cnc(N4CCN(C)CC4)nc23)c1F] | 1.06 | 479 |
| I-5 | 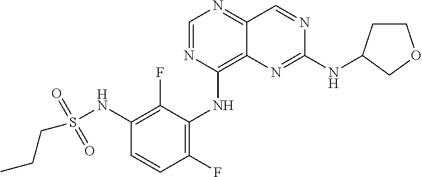 [SMILES: CCCS(=O)(=O)Nc1ccc(F)c(Nc2ncnc3cnc(NC4CCOC4)nc23)c1F] | 0.94 | 466 |
| I-6 | 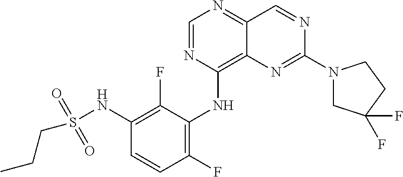 [SMILES: CCCS(=O)(=O)Nc1ccc(F)c(Nc2ncnc3cnc(N4CCC(F)(F)C4)nc23)c1F] | 1.17 | 486 |
| I-7 | 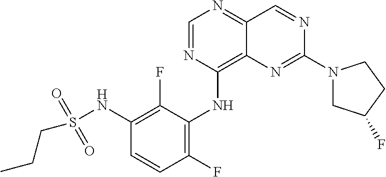 [SMILES: CCCS(=O)(=O)Nc1ccc(F)c(Nc2ncnc3cnc(N4CC[C@H](F)C4)nc23)c1F] | 1.33 | 468 |
| I-8 | 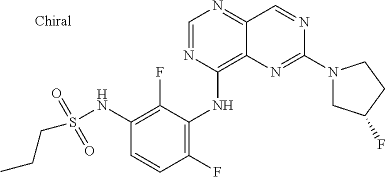 [SMILES: CCCS(=O)(=O)Nc1ccc(F)c(Nc2ncnc3cnc(N4CC[C@H](F)C4)nc23)c1F] | 1.10 | 468 |
| I-9 | 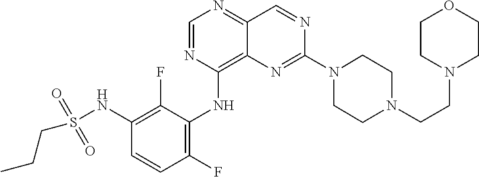 [SMILES: CCCS(=O)(=O)Nc1ccc(F)c(Nc2ncnc3cnc(N4CCN(CCN5CCOCC5)CC4)nc23)c1F] | 1.07 | 578 |
| I-10 | 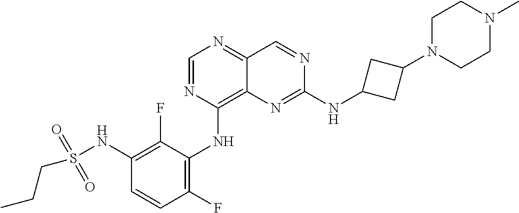 [SMILES: CCCS(=O)(=O)Nc1ccc(F)c(Nc2ncnc3cnc(NC4CC(N5CCN(C)CC5)C4)nc23)c1F] | 1.02 | 548 |
| I-11 | 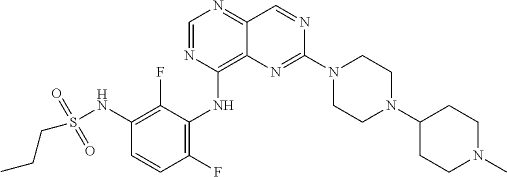 [SMILES: CCCS(=O)(=O)Nc1ccc(F)c(Nc2ncnc3cnc(N4CCN(C5CCN(C)CC5)CC4)nc23)c1F] | 1.16 | 562 |
| I-12 | 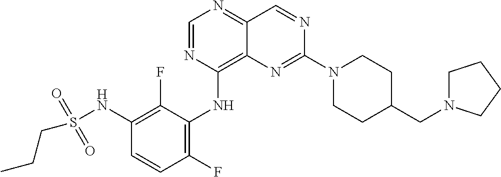 [SMILES: CCCS(=O)(=O)Nc1ccc(F)c(Nc2ncnc3cnc(N4CCC(CN5CCCC5)CC4)nc23)c1F] | 1.36 | 547 |
| I-13 | 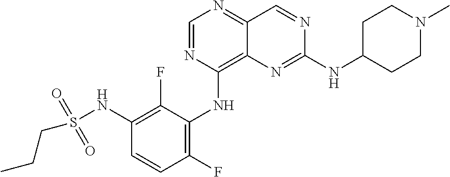 [SMILES: CCCS(=O)(=O)Nc1ccc(F)c(Nc2ncnc3cnc(NC4CCN(C)CC4)nc23)c1F] | 1.04 | 493 |
| I-14 | 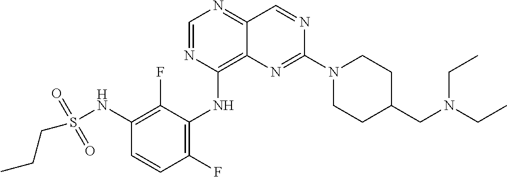 [SMILES: CCCS(=O)(=O)Nc1ccc(F)c(Nc2ncnc3cnc(N4CCC(CN(CC)CC)CC4)nc23)c1F] | 1.13 | 549 |
| I-15 | 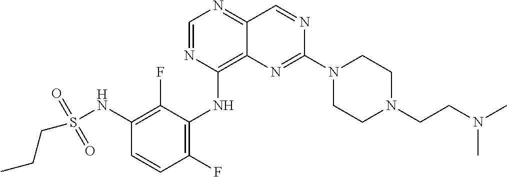 [SMILES: CCCS(=O)(=O)Nc1ccc(F)c(Nc2ncnc3cnc(N4CCN(CCN(C)C)CC4)nc23)c1F] | 0.89 | 536 |
| I-16 | 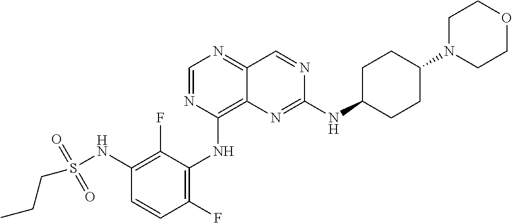 [SMILES: CCCS(=O)(=O)Nc1ccc(F)c(Nc2ncnc3cnc(N[C@H]4CC[C@H](N5CCOCC5)CC4)nc23)c1F] | 0.89 | 563 |
| I-17 | 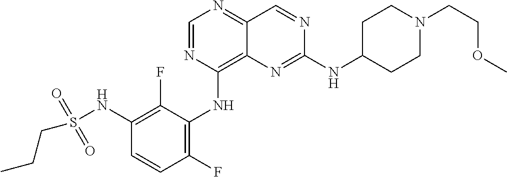 [SMILES: CCCS(=O)(=O)Nc1ccc(F)c(Nc2ncnc3cnc(NC4CCN(CCOC)CC4)nc23)c1F] | 0.88 | 537 |
| I-18 | 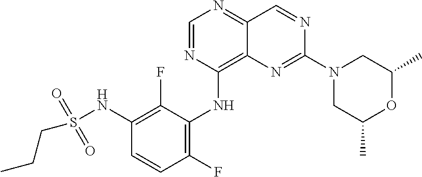 [SMILES: CCCS(=O)(=O)Nc1ccc(F)c(Nc2ncnc3cnc(N4C[C@@H](C)O[C@@H](C)C4)nc23)c1F] | 0.95 | 494 |
| I-19 | 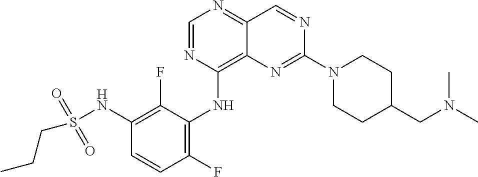 [SMILES: CCCS(=O)(=O)Nc1ccc(F)c(Nc2ncnc3cnc(N4CCC(CN(C)C)CC4)nc23)c1F] | 0.99 | 521 |
| I-20 | 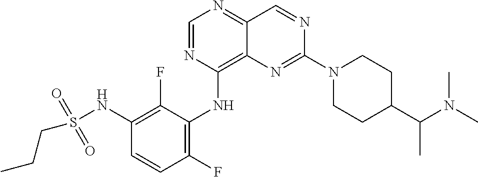 [SMILES: CCCS(=O)(=O)Nc1ccc(F)c(Nc2ncnc3cnc(N4CCC(C(C)N(C)C)CC4)nc23)c1F] | 1.05 | 535 |
| I-21 | 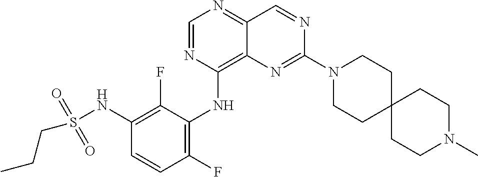 [SMILES: CCCS(=O)(=O)Nc1ccc(F)c(Nc2ncnc3cnc(N4CCC5(CCN(C)CC5)CC4)nc23)c1F] | 0.98 | 547 |
| I-22 | 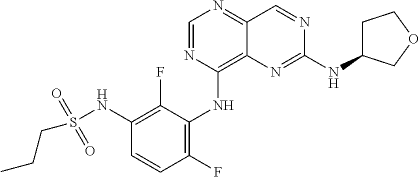 [SMILES: CCCS(=O)(=O)Nc1ccc(F)c(Nc2ncnc3cnc(N[C@H]4CCOC4)nc23)c1F] | 0.78 | 466 |
| I-23 | 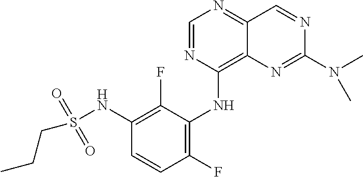 [SMILES: CCCS(=O)(=O)Nc1ccc(F)c(Nc2ncnc3cnc(N(C)C)nc23)c1F] | 0.85 | 424 |
| I-24 | 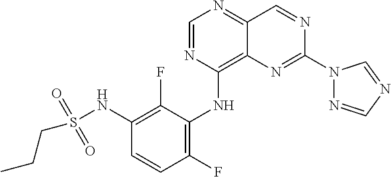 [SMILES: CCCS(=O)(=O)Nc1ccc(F)c(Nc2ncnc3cnc(-n4cncn4)nc23)c1F] | 0.00 | 446 |
| I-25 | 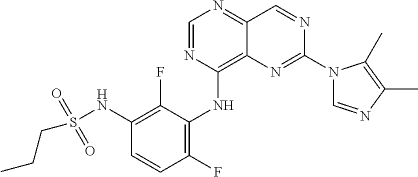 [SMILES: CCCS(=O)(=O)Nc1ccc(F)c(Nc2ncnc3cnc(-n4cnc(C)c4C)nc23)c1F] | 0.86 | 475 |
| I-26 | 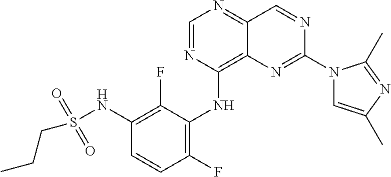 [SMILES: CCCS(=O)(=O)Nc1ccc(F)c(Nc2ncnc3cnc(-n4cc(C)nc4C)nc23)c1F] | 0.84 | 475 |
| I-27 | 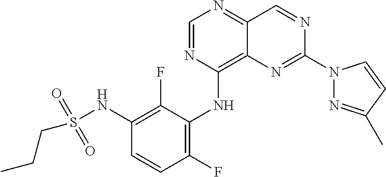 [SMILES: CCCS(=O)(=O)Nc1ccc(F)c(Nc2ncnc3cnc(-n4ccc(C)n4)nc23)c1F] | 0.85 | 461 |
| I-28 | 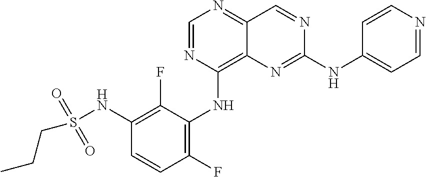 [SMILES: CCCS(=O)(=O)Nc1ccc(F)c(Nc2ncnc3cnc(Nc4ccncc4)nc23)c1F] | 0.00 | 473 |
| I-29 | 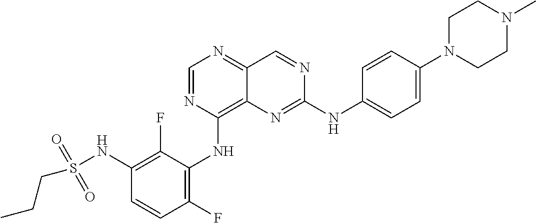 [SMILES: CCCS(=O)(=O)Nc1ccc(F)c(Nc2ncnc3cnc(Nc4ccc(N5CCN(C)CC5)cc4)nc23)c1F] | 0.91 | 570 |
| I-30 | 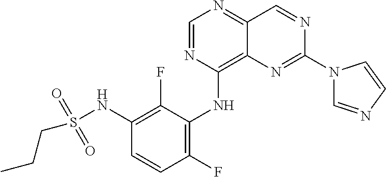 [SMILES: CCCS(=O)(=O)Nc1ccc(F)c(Nc2ncnc3cnc(-n4ccnc4)nc23)c1F] | 0.00 | 447 |
| I-31 | 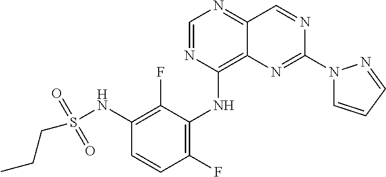 [SMILES: CCCS(=O)(=O)Nc1ccc(F)c(Nc2ncnc3cnc(-n4cccn4)nc23)c1F] | 0.78 | 447 |
| I-32 | 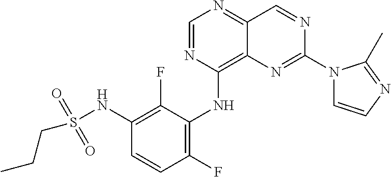 [SMILES: CCCS(=O)(=O)Nc1ccc(F)c(Nc2ncnc3cnc(-n4ccnc4C)nc23)c1F] | 0.77 | 461 |
| I-33 | 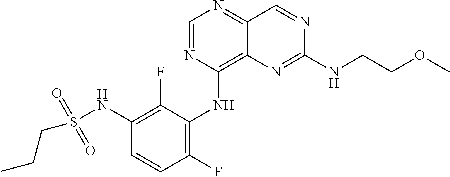 [SMILES: CCCS(=O)(=O)Nc1ccc(F)c(Nc2ncnc3cnc(NCCOC)nc23)c1F] | 0.76 | 454 |
| I-34 | 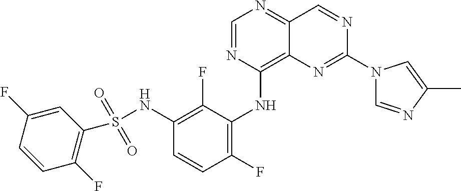 [SMILES: Cc1cn(-c2ncc3ncnc(Nc4c(F)ccc(NS(=O)(=O)c5cc(F)ccc5F)c4F)c3n2)cn1] | 0.97 | 531 |
| I-35 | 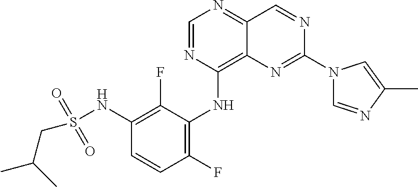 [SMILES: Cc1cn(-c2ncc3ncnc(Nc4c(F)ccc(NS(=O)(=O)CC(C)C)c4F)c3n2)cn1] | 0.92 | 475 |
| I-36 | 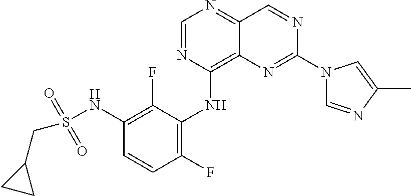 [SMILES: Cc1cn(-c2ncc3ncnc(Nc4c(F)ccc(NS(=O)(=O)CC5CC5)c4F)c3n2)cn1] | n.d. | 473 |

The following examples describe the biological activity of the compounds according to the invention without restricting the invention to these examples.

Compounds of general formula (I) are characterised by their many possible applications in the therapeutic field. Particular mention should be made of those applications in which the inhibition of specific signal enzymes, particularly the inhibiting effect on the proliferation of cultivated human tumour cells but also on the proliferation of other cells such as endothelial cells, for example, are involved.

### Kinase Test B-Raf (V600E)

In a dilution series 10 μL/well of test substance solution are placed in a multiwell plate. The dilution series is selected so that generally a range of concentrations of 2 μM to 0.119 nM or 0.017 nM is covered. If necessary the initial concentration of 2 μM is changed to 50 μM, 10 μM, 0.4 μM or 0.2857 μM and further dilution is carried out accordingly. The final concentration of DMSO is 5%. 10 μL/well of the B-Raf (V600E)-kinase solution are pipetted in (containing 0.5 ng B-Raf (V600E)-kinase, e.g. from Upstate) in 20 mM Tris-HCl pH 7.5, 0.1 mM EDTA, 0.1 mM EGTA, 0.286 mM sodium orthovanadate, 10% glycerol, 1 mg/mL bovine serum albumin, 1 mM dithiothreitol) and the mixture is incubated for 1 h at RT with shaking. The kinase reaction is started by the addition of 20 μL/well ATP solution [final concentration: 250 μM ATP, 30 mM Tris-HCl pH 7.5, 0.02% Brij, 0.2 mM sodium orthovanadate, 10 mM magnesium acetate, 0.1 mM EGTA, phosphatase cocktail (Sigma, # P2850, dilution recommended by the manufacturer)] and 10 μL/well MEK1 solution [containing 50 ng biotinylated MEK1 (prepared from purified MEK1 according to standard procedure, e.g. with EZ-Link Sulpho-NHS-LC-Biotin reagent, Pierce, #21335)] and carried out for 60 min at RT with constant shaking. The reaction is stopped by the addition of 12 μL/well of a 100 mM EDTA solution and incubation is continued for a further 5 min. 55 μL/well of the reaction solution are transferred into a streptavidin-coated plate (e.g. Streptawell HighBond, Roche, #11989685001) and gently shaken for 1 h at RT in order to bind biotinylated MEK1 to the plate. After elimination of the liquid the plate is washed five times with 200 μL/well of 1×PBS and 100 μL/well solution of primary antibody plus europium-labelled secondary antibody [Anti Phospho-MEK (Ser217/221), Cell Signaling, #9121 and Eu—N1 labelled goat-anti-rabbit antibody, Perkin Elmer, # AD0105] is added, the primary antibody is diluted 1:2000 and the secondary antibody is diluted to 0.4-0.5 μg/mL in Delfia Assay Buffer (Perkin Elmer, #1244-111). After 1 h shaking at RT the solution is poured away and washed five times with 200 μL/well Delfia Wash Buffer (Perkin Elmer, #4010-0010/#1244-114). After the addition of 200 μL/well Enhancement Solution (Perkin Elmer, #4001-0010/#1244-105) the mixture is shaken for 10 min at RT and then measured in a Wallac Victor using the program “Delfia Time Resolved Fluorescence (Europium)”. IC₅₀ values are obtained from these dosage-activity curves using a software program (GraphPadPrizm).

Measurement of the Inhibition of the Proliferation of Cultivated Human Melanoma Cells (SK-MEL-28, B-RAF^{V600E} mutated)

For measuring the proliferation of cultivated human tumour cells, cells of the melanoma cell line SK-MEL-28 [from American Type Culture Collection (ATCC)] are cultivated in MEM medium, supplemented with 10% foetal calf serum, 2% sodium bicarbonate, 1 mM sodium pyruvate, 1% non-essential amino acids (e.g. from Cambrex, # BE13-114E) and 2 mM glutamine. SK-MEL-28 cells are placed in 96-well flat bottomed dishes in a density of 2500 cells per well in supplemented MEM medium (see above) and incubated overnight in an incubator (at 37° C. and 5% CO₂). The active substances are added to the cells in different concentrations, so that a concentration range of 50 μM to 3.2 nM is covered. If necessary the initial concentration of 50 μM is changed to 10 μM or 2 μM and further dilution is carried out accordingly (up to 0.6 nM or 0.12 nM). After an incubation period of a further 72 h 20 μL AlamarBlue reagent (Serotec Ltd., # BUF012B) are added to each well and the cells are incubated for a further 3-6 h. The colour change of the AlamarBlue reagent is determined in a fluorescence spectrophotometer (e.g. Gemini, Molecular Devices). EC₅₀ values are calculated using a software program (GraphPadPrizm).

The EC₅₀ values of the example compounds determined using the above assay are shown in Table 3.

TABLE 3

| | EC₅₀ SK-MEL- |
|---|---|
| # | 28 [nM] |
| I-1 | 276 |
| I-2 | 514 |
| I-3 | 1192 |
| I-4 | 1480 |
| I-5 | 668 |
| I-6 | 493 |
| I-7 | 176 |
| I-8 | 522 |
| I-9 | 1136 |
| I-10 | 1273 |
| I-11 | 1070 |
| I-12 | 952 |
| I-13 | 1482 |
| I-14 | 1670 |
| I-15 | 781 |
| I-16 | 1029 |
| I-17 | 1596 |
| I-18 | 774 |
| I-19 | 1252 |
| I-20 | 1594 |
| I-21 | 440 |
| I-22 | 919 |
| I-23 | 1631 |
| I-24 | 1501 |
| I-25 | 143 |
| I-26 | 212 |
| I-27 | 807 |
| I-28 | 424 |
| I-29 | 526 |
| I-30 | 504 |
| I-31 | 936 |
| I-32 | 297 |
| I-33 | 1805 |

Measurement of the Inhibition of the Proliferation of Cultivated Human Melanoma Cells (A375, B-RAF^{V600E} mutated)

For measuring the proliferation of cultivated human tumour cells, cells of the melanoma cell line A375 [from the American Type Culture Collection (ATCC)] are cultivated in DMEM medium, supplemented with 10% foetal calf serum and 2% sodium bicarbonate. Test substances are tested on A375 cells according to the procedure described for SK-MEL-28 cells (see above), but seeding them at 5000 cells per well.

Most of the example compounds I-1 to I-36 show good to very good activity in the cellular A375 assay, i.e. an EC₅₀ value of less than ca. 1000 nM, often less than 500 nM.

The active substances are characterised in that they have a significantly lower antiproliferative activity on cell lines which have no B-RAF mutation. Thus, for example, example compounds I-1 to I-36 have an EC₅₀ value on melanoma cells (e.g. A375) without a B-Raf V600E mutation which is generally higher than that of B-RAF mutated melanoma cells (e.g. A375) by at least a factor of 100.

The EC₅₀ value of the phospho-ERK reduction and the EC₅₀ value of the antiproliferative activity in B-RAF mutated cell lines correlate well with cellular selectivity of the active substances.

### Measurement of the Reduction of the Phospho-Erk Signal in Cultivated Human Melanoma Cells (SK-MEL-28, B-RAF^{V600E} Mutated)

To measure the reduction in the phospho-ERK signal of cultivated human tumour cells, cells of the melanoma cell line SK-MEL-28 [from the American Type Culture Collection (ATCC)] in MEM medium, supplemented with 10% foetal calf serum, 2% sodium bicarbonate, 1 mM sodium pyruvate, 1% non-essential amino acids (e.g. obtained from Cambrex, # BE13-114E) and 2 mM glutamine, are cultivated. SK-MEL-28 cells are placed in 96-well flat bottomed dishes in a density of 7500 cells per well in supplemented MEM medium (see above) and incubated overnight in an incubator (at 37° C. and 5% CO₂). The active substances are added to the cells in different concentrations, so that a concentration range of 10 μM to 2.4 nM is covered. If necessary the initial concentration of 10 μM is changed to 50 11M or 2.5 11M and further dilution is carried out accordingly (up to 12.2 nM or 0.6 nM). After an incubation period of a further 2 h the cells are fixed with 4 formaldehyde and permeabilised with 0.1% Triton X-100 in PBS. Non-specific antibody binding is reduced by incubating with 5% skimmed milk powder dissolved in TBS-T. Phosphorylated ERK is detected with a murine monoclonal anti-diphosphorylated ERK1/2 antibody (from Sigma, #M8159). After washing steps using 0.1% Tween 20 in PBS the bound first antibody is detected by the second antibody (peroxidase coupled polyclonal rabbit anti mouse IgG from DAKO #P0161). After further washing steps the substrate (TMB Peroxidase Substrate Solution made by Bender MedSystems #BMS406) is added. The colour reaction is stopped after a few minutes with 1M phosphoric acid. The staining is measured at 450 nm with a Spectra Max Plus reader made by Molecular Devices. EC₅₀ values are calculated using a software program (GraphPadPrizm).

The EC₅₀ value of the phospho-ERK reduction of the example compounds determined using the above assay is generally less than 500 nM.

The substances of the present invention are B-RAF-kinase inhibitors. As can be demonstrated by DNA staining followed by FACS or Cellomics Array Scan analysis, the inhibition of proliferation achieved by means of the compounds according to the invention is brought about above all by preventing entry into the DNA synthesis phase. The treated cells arrest in the G1 phase of the cell cycle.

Accordingly, the compounds according to the invention are also tested on other tumour cells. For example these compounds are effective on colon carcinoma lines, e.g. Colo205, HT29, and may be used in this and other indications. This demonstrates the usefulness of the compounds according to the invention for the treatment of different types of tumours.

On the basis of their biological properties the compounds of general formula (I) according to the invention, their tautomers, racemates, enantiomers, diastereomers, mixtures thereof and the salts of all the above-mentioned forms are suitable for treating diseases characterised by excessive or abnormal cell proliferation.

Such diseases include for example: viral infections (e.g. HIV and Kaposi's sarcoma); inflammatory and autoimmune diseases (e.g. colitis, arthritis, Alzheimer's disease, glomerulonephritis and wound healing); bacterial, fungal and/or parasitic infections; leukaemias, lymphomas and solid tumours (e.g. carcinomas and sarcomas), skin diseases (e.g. psoriasis); diseases based on hyperplasia which are characterised by an increase in the number of cells (e.g. fibroblasts, hepatocytes, bones and bone marrow cells, cartilage or smooth muscle cells or epithelial cells (e.g. endometrial hyperplasia); bone diseases and cardiovascular diseases (e.g. restenosis and hypertrophy). They are also suitable for protecting proliferating cells (e.g. hair, intestinal, blood and progenitor cells) from DNA damage caused by radiation, UV treatment and/or cytostatic treatment.

For example, the following cancers may be treated with compounds according to the invention, without being restricted thereto:

brain tumours such as for example acoustic neurinoma, astrocytomas such as pilocytic astrocytomas, fibrillary astrocytoma, protoplasmic astrocytoma, gemistocytary astrocytoma, anaplastic astrocytoma and glioblastoma, brain lymphomas, brain metastases, hypophyseal tumour such as prolactinoma, HGH (human growth hormone) producing tumour and ACTH producing tumour (adrenocorticotropic hormone), craniopharyngiomas, medulloblastomas, meningeomas and oligodendrogliomas; nerve tumours (neoplasms) such as for example tumours of the vegetative nervous system such as neuroblastoma sympathicum, ganglioneuroma, paraganglioma (pheochromocytoma, chromaffinoma) and glomus-caroticum tumour, tumours on the peripheral nervous system such as amputation neuroma, neurofibroma, neurinoma (neurilemmoma, Schwannoma) and malignant Schwannoma, as well as tumours of the central nervous system such as brain and bone marrow tumours; intestinal cancer such as for example carcinoma of the rectum, colon carcinoma, colorectal carcinoma, anal carcinoma, carcinoma of the large bowel, tumours of the small intestine and duodenum; eyelid tumours such as basalioma or basal cell carcinoma; pancreatic cancer or carcinoma of the pancreas; bladder cancer or carcinoma of the bladder; lung cancer (bronchial carcinoma) such as for example small-cell bronchial carcinomas (oat cell carcinomas) and non-small cell bronchial carcinomas (NSCLC) such as plate epithelial carcinomas, adenocarcinomas and large-cell bronchial carcinomas; breast cancer such as for example mammary carcinoma such as infiltrating ductal carcinoma, colloid carcinoma, lobular invasive carcinoma, tubular carcinoma, adenocystic carcinoma and papillary carcinoma; non-Hodgkin's lymphomas (NHL) such as for example Burkitt's lymphoma, low-malignancy non-Hodgkin's lymphomas (NHL) and mucosis fungoides; uterine cancer or endometrial carcinoma or corpus carcinoma; CUP syndrome (Cancer of Unknown Primary); ovarian cancer or ovarian carcinoma such as mucinous, endometrial or serous cancer; gall bladder cancer; bile duct cancer such as for example Klatskin tumour; testicular cancer such as for example seminomas and non-seminomas; lymphoma (lymphosarcoma) such as for example malignant lymphoma, Hodgkin's disease, non-Hodgkin's lymphomas (NHL) such as chronic lymphatic leukaemia, leukaemic reticuloendotheliosis, immunocytoma, plasmocytoma (multiple myeloma), immunoblastoma, Burkitt's lymphoma, T-zone mycosis fungoides, large-cell anaplastic lymphoblastoma and lymphoblastoma; laryngeal cancer such as for example tumours of the vocal cords, supraglottal, glottal and subglottal laryngeal tumours; bone cancer such as for example osteochondroma, chondroma, chondroblastoma, chondromyxoid fibroma, osteoma, osteoid osteoma, osteoblastoma, eosinophilic granuloma, giant cell tumour, chondrosarcoma, osteosarcoma, Ewing's sarcoma, reticulo-sarcoma, plasmocytoma, fibrous dysplasia, juvenile bone cysts and aneurysmatic bone cysts; head and neck tumours such as for example tumours of the lips, tongue, floor of the mouth, oral cavity, gums, palate, salivary glands, throat, nasal cavity, paranasal sinuses, larynx and middle ear; liver cancer such as for example liver cell carcinoma or hepatocellular carcinoma (HCC); leukaemias, such as for example acute leukaemias such as acute lymphatic/lymphoblastic leukaemia (ALL), acute myeloid leukaemia (AML); chronic leukaemias such as chronic lymphatic leukaemia (CLL), chronic myeloid leukaemia (CML); stomach cancer or gastric carcinoma such as for example papillary, tubular and mucinous adenocarcinoma, signet ring cell carcinoma, adenosquamous carcinoma, small-cell carcinoma and undifferentiated carcinoma; melanomas such as for example superficially spreading, nodular, lentigo-maligna and acral-lentiginous melanoma; renal cancer such as for example kidney cell carcinoma or hypernephroma or Grawitz's tumour; oesophageal cancer or carcinoma of the oesophagus; penile cancer; prostate cancer; throat cancer or carcinomas of the pharynx such as for example nasopharynx carcinomas, oropharynx carcinomas and hypopharynx carcinomas; retinoblastoma, vaginal cancer or vaginal carcinoma; plate epithelial carcinomas, adenocarcinomas, in situ carcinomas, malignant melanomas and sarcomas; thyroid carcinomas such as for example papillary, follicular and medullary thyroid carcinoma, as well as anaplastic carcinomas; spinalioma, epidormoid carcinoma and plate epithelial carcinoma of the skin; thymomas, cancer of the urethra and cancer of the vulva.

The new compounds may be used for the prevention, short-term or long-term treatment of the above-mentioned diseases, optionally also in combination with radiotherapy or other “state-of-the-art” compounds, such as e.g. cytostatic or cytotoxic substances, cell proliferation inhibitors, anti-angiogenic substances, steroids or antibodies.

The compounds of general formula (I) may be used on their own or in combination with other active substances according to the invention, optionally also in combination with other pharmacologically active substances.

Chemotherapeutic agents which may be administered in combination with the compounds according to the invention, include, without being restricted thereto, hormones, hormone analogues and antihormones (e.g. tamoxifen, toremifene, raloxifene, fulvestrant, megestrol acetate, flutamide, nilutamide, bicalutamide, aminoglutethimide, cyproterone acetate, finasteride, buserelin acetate, fludrocortisone, fluoxymesterone, medroxy-progesterone, octreotide), aromatase inhibitors (e.g. anastrozole, letrozole, liarozole, vorozole, exemestane, atamestane), LHRH agonists and antagonists (e.g. goserelin acetate, luprolide), inhibitors of growth factors (growth factors such as for example “platelet derived growth factor (PDGF)”, “fibroblast growth factor (FGF)”, “vascular endothelial growth factor (VEGF)”, “epidermal growth factor (EGF)”, “insuline-like growth factors (IGF)”, “human epidermal growth factor (HER, e.g. HER2, HER3, HER4)” and “hepatocyte growth factor (HGF)”), inhibitors are for example “growth factor” antibodies, “growth factor receptor” antibodies and tyrosine kinase inhibitors, such as for example cetuximab, gefitinib, imatinib, lapatinib and trastuzumab); antimetabolites (e.g. antifolates such as methotrexate, raltitrexed, pyrimidine analogues such as 5-fluorouracil, capecitabin and gemcitabin, purine and adenosine analogues such as mercaptopurine, thioguanine, cladribine and pentostatin, cytarabine, fludarabine); antitumour antibiotics (e.g. anthracyclins such as doxorubicin, daunorubicin, epirubicin and idarubicin, mitomycin-C, bleomycin, dactinomycin, plicamycin, streptozocin); platinum derivatives (e.g. cisplatin, oxaliplatin, carboplatin); alkylation agents (e.g. estramustin, meclorethamine, melphalan, chlorambucil, busulphan, dacarbazin, cyclophosphamide, ifosfamide, temozolomide, nitrosoureas such as for example carmustin and lomustin, thiotepa); antimitotic agents (e.g. Vinca alkaloids such as for example vinblastine, vindesin, vinorelbin and vincristine; and taxanes such as paclitaxel, docetaxel); tubuline inhibitors; PARP inhibitors, topoisomerase inhibitors (e.g. epipodophyllotoxins such as for example etoposide and etopophos, teniposide, amsacrin, topotecan, irinotecan, mitoxantron), serine/threonine kinase inhibitors (e.g. PDK 1 inhibitors, B-Raf inhibitors, mTOR inhibitors, mTORC1 inhibitors, PI3K inhibitors, dual mTOR/PI3K inhibitors, STK 33 inhibitors, AKT inhibitors, PLK 1 inhibitors, inhibitors of CDKs, Aurora kinase inhibitors), tyrosine kinase inhibitors (e.g. PTK2/FAK inhibitors), protein protein interaction inhibitors (e.g. IAP, Mc1-1, MDM2/MDMX), MEK inhibitors, ERK inhibitors, IGF-1R inhibitors, ErbB receptor inhibitors, rapamycin analogs (e.g. everolimus, temsirolimus, ridaforolimus, sirolimus) and various chemotherapeutic agents such as amifostin, anagrelid, clodronat, filgrastin, interferon, interferon alpha, leucovorin, rituximab, procarbazine, levamisole, mesna, mitotane, pamidronate and porfimer.

Other possible combination partners are 2-chlorodesoxyadenosine, 2-fluorodesoxy-cytidine, 2-methoxyoestradiol, 2C4,3-alethine, 131-I-TM-601, 3CPA, 7-ethyl-10-hydroxycamptothecin, 16-aza-epothilone B, A 105972, A 204197, abiraterone, aldesleukin, alitretinoin, allovectin-7, altretamine, alvocidib, amonafide, anthrapyrazole, AG-2037, AP-5280, apaziquone, apomine, aranose, arglabin, arzoxifene, atamestane, atrasentan, auristatin PE, AVLB, AZ10992, ABX-EGF, AMG-479 (ganitumab), ARRY 162, ARRY 438162, ARRY-300, ARRY-142886/AZD-6244 (selumetinib), ARRY-704/AZD-8330, AR-12, AR-42, AS-703988, AXL-1717, AZD-8055, AZD-5363, AZD-6244, ARQ-736, ARQ 680, AS-703026 (primasertib), avastin, AZD-2014, azacytidine, azaepothilone B, azonafide, BAY-43-9006, BAY 80-6946, BBR-3464, BBR-3576, bevacizumab, BEZ-235, biricodar dicitrate, BCX-1777, BKM-120, bleocin, BLP-25, BMS-184476, BMS-247550, BMS-188797, BMS-275291, BMS-663513, BMS-754807, BNP-1350, BNP-7787, BIBW 2992 (afatinib, tomtovok), BIBF 1120 (vargatef), BI 836845, BI 2536, BI 6727, BI 836845, BI 847325, BI 853520, BIIB-022, bleomycinic acid, bleomycin A, bleomycin B, brivanib, bryostatin-1, bortezomib, brostallicin, busulphan, BYL-719, CA-4 prodrug, CA-4, CapCell, calcitriol, canertinib, canfosfamide, capecitabine, carboxyphthalatoplatin, CCI-779, CC-115, CC-223, CEP-701, CEP-751, CBT-1 cefixime, ceflatonin, ceftriaxone, celecoxib, celmoleukin, cemadotin, CH4987655/RO-4987655, chlorotrianisene, cilengitide, ciclosporin, CDA-II, CDC-394, CKD-602, CKI-27, clofarabin, colchicin, combretastatin A4, COT inhibitors, CHS-828, CH-5132799, CLL-Thera, CMT-3 cryptophycin 52, CTP-37, CTLA-4 monoclonal antibodies, CP-461, CV-247, cyanomorpholinodoxorubicin, cytarabine, D 24851, decitabine, deoxorubicin, deoxyrubicin, deoxycoformycin, depsipeptide, desoxyepothilone B, dexamethasone, dexrazoxanet, diethylstilbestrol, diflomotecan, didox, DMDC, dolastatin 10, doranidazole, DS-7423, E7010, E-6201, edatrexat, edotreotide, efaproxiral, eflornithine, EGFR inhibitors, EKB-569, EKB-509, enzastaurin, elsamitrucin, epothilone B, epratuzumab, ER-86526, erlotinib, ET-18-OCH3, ethynylcytidine, ethynyloestradiol, exatecan, exatecan mesylate, exemestane, exisulind, fenretinide, figitumumab, floxuridine, folic acid, FOLFOX, FOLFOX4, FOLFIR1, formestane, fotemustine, galarubicin, gallium maltolate, gefinitib, gemtuzumab, gimatecan, glufosfamide, GCS-100, GDC-0623, GDC-0941 (pictrelisib), GDC-0980, GDC-0032, GDC-0068, GDC-0349, GDC-0879, G17DT immunogen, GMK, GPX-100, gp100-peptide vaccines, GSK-5126766, GSK-690693, GSK-1120212 (trametinib), GSK-2118436 (dabrafenib), GSK-2126458, GSK-2132231A, GSK-2334470, GSK-2110183, GSK-2141795, GW2016, granisetron, herceptine, hexamethylmelamine, histamine, homoharringtonine, hyaluronic acid, hydroxyurea, hydroxyprogesterone caproate, ibandronate, ibritumomab, idatrexate, idenestrol, IDN-5109, IGF-1R inhibitors, IMC-1C11, IMC-A12 (cixutumumab), immunol, indisulam, interferon alpha-2a, interferon alpha-2b, pegylated interferon alpha-2b, interleukin-2, INK-1117, INK-128, INSM-18, ionafarnib, ipilimumab, iproplatin, irofulven, isohomohalichondrin-B, isoflavone, isotretinoin, ixabepilone, JRX-2, JSF-154, J-107088, conjugated oestrogens, kahalid F, ketoconazole, KW-2170, KW-2450, lobaplatin, leflunomide, lenograstim, leuprolide, leuporelin, lexidronam, LGD-1550, linezolid, lutetium texaphyrin, lometrexol, losoxantrone, LU 223651, lurtotecan, LY-S6AKT1, LY-2780301, mafosfamide, marimastat, mechloroethamine, MEK inhibitors, MEK-162, methyltestosteron, methylprednisolone, MEDI-573, MEN-10755, MDX-H210, MDX-447, MDX-1379, MGV, midostaurin, minodronic acid, mitomycin, mivobulin, MK-2206, MK-0646 (dalotuzumab), MLN518, motexaf in gadolinium, MS-209, MS-275, MX6, neridronate, neratinib, Nexavar, neovastat, nilotinib, nimesulide, nitroglycerin, nolatrexed, norelin, N-acetylcysteine, 06-benzylguanine, oblimersen, omeprazole, oncophage, oncoVEX^{Gm-CSF}, ormiplatin, ortataxel, OX44 antibodies, OSI-027, OSI-906 (linsitinib), 4-1BB antibodies, oxantrazole, oestrogen, panitumumab, patupilone, pegfilgrastim, PCK-3145, pegfilgrastim, PBI-1402, PBI-05204, PDO325901, PD-1 antibodies, PEG-paclitaxel, albumin-stabilized paclitaxel, PEP-005, PF-05197281, PF-05212384, PF-04691502, PHT-427, P-04, PKC412, P54, PI-88, pelitinib, pemetrexed, pentrix, perifosine, perillylalcohol, pertuzumab, PI3K inhibitors, PI3K/mTOR inhibitors, PG-TXL, PG2, PLX-4032/RO-5185426 (vemurafenib), PLX-3603/RO-5212054, PT-100, PWT-33597, PX-866, picoplatin, pivaloyloxymethylbutyrate, pixantrone, phenoxodiol O, PKI166, plevitrexed, plicamycin, polyprenic acid, porfiromycin, prednisone, prednisolone, quinamed, quinupristin, R115777, RAF-265, ramosetron, ranpirnase, RDEA-119/BAY 869766, RDEA-436, rebeccamycin analogues, receptor tyrosine kinase (RTK) inhibitors, revimid, RG-7167, RG-7304, RG-7421, RG-7321, RG 7440, rhizoxin, rhu-MAb, rinfabate, risedronate, rituximab, robatumumab, rofecoxib, RO-31-7453, RO-5126766, RO-5068760, RPR 109881A, rubidazone, rubitecan, R-flurbiprofen, RX-0201, S-9788, sabarubicin, SAHA, sargramostim, satraplatin, SB 408075, Se-015/Ve-015, SU5416, SU6668, SDX-101, semustin, seocalcitol, SM-11355, SN-38, SN-4071, SR-27897, SR-31747, SR-13668, SRL-172, sorafenib, spiroplatin, squalamine, suberanilohydroxamic acid, sutent, T 900607, T 138067, TAK-733, TAS-103, tacedinaline, talaporf in, Tarceva, tariquitar, tasisulam, taxotere, taxoprexin, tazarotene, tegafur, temozolamide, tesmilifene, testosterone, testosterone propionate, tesmilifene, tetraplatin, tetrodotoxin, tezacitabine, thalidomide, theralux, therarubicin, thymalfasin, thymectacin, tiazofurin, tipifarnib, tirapazamine, tocladesine, tomudex, toremofin, trabectedin, TransMID-107, transretinic acid, traszutumab, tremelimumab, tretinoin, triacetyluridine, triapine, triciribine, trimetrexate, TLK-286TXD 258, tykerb/tyverb, urocidin, valrubicin, vatalanib, vincristine, vinflunine, virulizin, WX-UK1, WX-554, vectibix, xeloda, XELOX, XL-147, XL-228, XL-281, XL-518/R-7420/GDC-0973, XL-765, YM-511, YM-598, ZD-4190, ZD-6474, ZD-4054, ZD-0473, ZD-6126, ZD-9331, ZD1839, ZSTK-474, zoledronat and zosuquidar.

Suitable preparations include for example tablets, capsules, suppositories, solutions—particularly solutions for injection (s.c., i.v., i.m.) and infusion—elixirs, emulsions or dispersible powders. The content of the pharmaceutically active compound(s) should be in the range from 0.1 to 90 wt.-%, preferably 0.5 to 50 wt.-% of the composition as a whole, i.e. in amounts which are sufficient to achieve the dosage range specified below. The doses specified may, if necessary, be given several times a day.

Suitable tablets may be obtained, for example, by mixing the active substance(s) with known excipients, for example inert diluents such as calcium carbonate, calcium phosphate or lactose, disintegrants such as corn starch or alginic acid, binders such as starch or gelatine, lubricants such as magnesium stearate or talc and/or agents for delaying release, such as carboxymethyl cellulose, cellulose acetate phthalate, or polyvinyl acetate. The tablets may also comprise several layers.

Coated tablets may be prepared accordingly by coating cores produced analogously to the tablets with substances normally used for tablet coatings, for example collidone or shellac, gum arabic, talc, titanium dioxide or sugar. To achieve delayed release or prevent incompatibilities the core may also consist of a number of layers. Similarly the tablet coating may consist of a number of layers to achieve delayed release, possibly using the excipients mentioned above for the tablets.

Syrups or elixirs containing the active substances or combinations thereof according to the invention may additionally contain a sweetener such as saccharine, cyclamate, glycerol or sugar and a flavour enhancer, e.g. a flavouring such as vanillin or orange extract. They may also contain suspension adjuvants or thickeners such as sodium carboxymethyl cellulose, wetting agents such as, for example, condensation products of fatty alcohols with ethylene oxide, or preservatives such as p-hydroxybenzoates.

Solutions for injection and infusion are prepared in the usual way, e.g. with the addition of isotonic agents, preservatives such as p-hydroxybenzoates, or stabilisers such as alkali metal salts of ethylenediamine tetraacetic acid, optionally using emulsifiers and/or dispersants, whilst if water is used as the diluent, for example, organic solvents may optionally be used as solvating agents or dissolving aids, and transferred into injection vials or ampoules or infusion bottles.

Capsules containing one or more active substances or combinations of active substances may for example be prepared by mixing the active substances with inert carriers such as lactose or sorbitol and packing them into gelatine capsules.

Suitable suppositories may be made for example by mixing with carriers provided for this purpose such as neutral fats or polyethyleneglycol or the derivatives thereof.

Excipients which may be used include, for example, water, pharmaceutically acceptable organic solvents such as paraffins (e.g. petroleum fractions), vegetable oils (e.g. groundnut or sesame oil), mono- or polyfunctional alcohols (e.g. ethanol or glycerol), carriers such as e.g. natural mineral powders (e.g. kaolins, clays, talc, chalk), synthetic mineral powders (e.g. highly dispersed silicic acid and silicates), sugars (e.g. cane sugar, lactose and glucose), emulsifiers (e.g. lignin, spent sulphite liquors, methylcellulose, starch and polyvinylpyrrolidone) and lubricants (e.g. magnesium stearate, talc, stearic acid and sodium lauryl sulphate).

The preparations are administered by the usual methods, preferably by oral or transdermal route, most preferably by oral route. For oral administration the tablets may of course contain, apart from the above-mentioned carriers, additives such as sodium citrate, calcium carbonate and dicalcium phosphate together with various additives such as starch, preferably potato starch, gelatine and the like. Moreover, lubricants such as magnesium stearate, sodium lauryl sulphate and talc may be used at the same time for the tabletting process. In the case of aqueous suspensions the active substances may be combined with various flavour enhancers or colourings in addition to the excipients mentioned above.

For parenteral use, solutions of the active substances with suitable liquid carriers may be used.

The dosage for intravenous use is from 1-1000 mg per hour, preferably between 5 and 500 mg per hour.

However, it may sometimes be necessary to depart from the amounts specified, depending on the body weight, the route of administration, the individual response to the drug, the nature of its formulation and the time or interval over which the drug is administered. Thus, in some cases it may be sufficient to use less than the minimum dose given above, whereas in other cases the upper limit may have to be exceeded. When administering large amounts it may be advisable to divide them up into a number of smaller doses spread over the day.

The formulation examples which follow illustrate the present invention without restricting its scope:

### Examples of Pharmaceutical Formulations

| | | |
|---|---|---|
| A) | Tablets | per tablet |

| | |
|---|---|
| active substance according to formula (I) | 100 mg |
| lactose | 140 mg |
| corn starch | 240 mg |
| polyvinylpyrrolidone | 15 mg |
| magnesium stearate | 5 mg |
| | 500 mg |

The finely ground active substance, lactose and some of the corn starch are mixed together. The mixture is screened, then moistened with a solution of polyvinylpyrrolidone in water, kneaded, wet-granulated and dried. The granules, the remaining corn starch and the magnesium stearate are screened and mixed together. The mixture is compressed to produce tablets of suitable shape and size.

| | | |
|---|---|---|
| B) | Tablets | per tablet |

| | |
|---|---|
| active substance according to formula (I) | 80 mg |
| lactose | 55 mg |
| corn starch | 190 mg |
| microcrystalline cellulose | 35 mg |
| polyvinylpyrrolidone | 15 mg |
| sodiumcarboxymethyl starch | 23 mg |
| magnesium stearate | 2 mg |
| | 400 mg |

The finely ground active substance, some of the corn starch, lactose, microcrystalline cellulose and polyvinylpyrrolidone are mixed together, the mixture is screened and worked with the remaining corn starch and water to form a granulate which is dried and screened. The sodiumcarboxymethyl starch and the magnesium stearate are added and mixed in and the mixture is compressed to form tablets of a suitable size.

| | |
|---|---|
| C) | Ampoule solution |

| | |
|---|---|
| active substance according to formula (I) | 50 mg |
| sodium chloride | 50 mg |
| water for inj. | 5 mL |

The active substance is dissolved in water at its own pH or optionally at pH 5.5 to 6.5 and sodium chloride is added to make it isotonic. The solution obtained is filtered free from pyrogens and the filtrate is transferred under aseptic conditions into ampoules which are then sterilised and sealed by fusion. The ampoules contain 5 mg, 25 mg and 50 mg of active substance.

1. A compound of the formula (I) 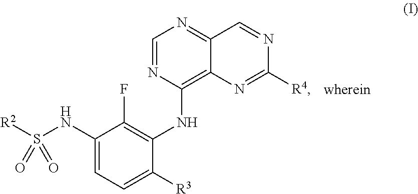
R² is a group optionally substituted by one or more, identical or different R^{b1} and/or R^{c1}, selected from among C₁₋₆alkyl, C₂₋₆alkenyl, C₂₋₆alkynyl, C₁₋₆haloalkyl, C₃₋₆cycloalkyl, C₄₋₆cycloalkenyl, C₆₋₁₀aryl, 5-10 membered heteroaryl and 3-10 membered heterocyclyl or
R² is —NR^{c1}R^{c1};
each R^{b1} is independently selected from among —OR^{c1}, —NR^{c1}R^{c1}, halogen, —CN, —C(O)R^{c1}, —C(O)OR^{c1}, —C(O)NR^{c1}R^{c1}, —S(O)₂R^{c1}, —S(O)₂NR^{c1}R^{c1}, —NHC(O)R^{c1} and —N(C₁₋₄alkyl)C(O)R^{c1} as well as the bivalent substituent ═O, wherein the latter may only be a substituent in non-aromatic ring systems;
each R^{c1} independently of one another denotes hydrogen or a group selected from among C₁₋₆alkyl, C₂₋₆alkenyl, C₂₋₆alkynyl, C₁₋₆haloalkyl, C₃₋₆cycloalkyl, C₄₋₆cycloalkenyl, C₆₋₁₀aryl, 5-10 membered heteroaryl and 3-10 membered heterocyclyl;
R³ is selected from among hydrogen, halogen, C₁₋₄alkyl, C₁₋₄alkyloxy, C₂₋₄alkenyl, C₂₋₄alkynyl, C₁₋₄haloalkyl, —CN, —NH(C₁₋₄alkyl) and —N(C₁₋₄alkyl)₂;
R⁴ denotes a group optionally substituted by one or more, identical or different R^{a2} and/or R^{b2}, selected from among C₁₋₆alkyl, C₂₋₆alkenyl, C₂₋₆alkynyl, C₁₋₆haloalkyl, C₃₋₆cycloalkyl, C₄₋₆cycloalkenyl, C₆₋₁₀aryl, 5-10 membered heteroaryl and 3-11 membered heterocyclyl, or is selected from among —OR^{a3}, —NR^{a3}R^{a3}, —N(OR^{a3})R^{a3}, —CN, —C(O)R^{a3}, —C(O)OR^{a3}, —C(O)NR^{a3}R^{a3}, —C(NH)NR^{a3}R^{a3}, —S(O)₂NR^{a3}R^{a3}, —NHS(O)₂R^{a3}, —N(C₁₋₄ alkyl)S(O)₂R^{a3}, —NHS(O)₂NR^{a3}R^{a3}, —NHC(O)R^{a3}, —N(C₁₋₄alkyl)C(O)R^{a3}, —NHC(O)OR^{a3}, —N(C₁₋₄alkyl)C(O)OR^{a3}, —NHC(O)NR^{a3}R^{a3} and —N(C₁₋₄alkyl)C(O)NR^{a3}R^{a3};
each R^{a2} independently of one another denotes a group optionally substituted by one or more, identical or different R^{b2} and/or R^{c2}, selected from among C₁₋₆alkyl, C₂₋₆alkenyl, C₂₋₆alkynyl, C₁₋₆haloalkyl, C₃₋₆cycloalkyl, C₄₋₆cycloalkenyl and 3-10 membered heterocyclyl;
each R^{b2} is independently selected from among —OR^{c2}, —NR^{c2}R^{c2}, halogen, —C(O) R^{c2}, —C(O)OR^{c2}, —C(O)NR^{c2}R^{c2}, —CN, —NHC(O)R^{c2} and —NHC(O)OR^{c2};
each R^{c2} independently of one another denotes hydrogen or a group selected from among C₁₋₆alkyl, C₂₋₆alkenyl, C₂₋₆alkynyl, C₃₋₆cycloalkyl, C₄₋₆cycloalkenyl and 3-10 membered heterocyclyl, wherein this heterocyclyl is optionally substituted by one or more, identical or different substituents selected from among halogen, C₁₋₆alkyl and —C(O)—C₁₋₆alkyl;
each R^{a3} independently of one another denotes hydrogen or a group optionally substituted by one or more, identical or different R^{b3} and/or R^{c3}, selected from among C₁₋₆alkyl, C₂₋₆alkenyl, C₂₋₆alkynyl, C₁₋₆haloalkyl, C₃₋₆cycloalkyl, C₄₋₆cycloalkenyl, C₆₋₁₀aryl, 5-10 membered heteroaryl and 3-10 membered heterocyclyl;
each R^{b3} is independently selected from among —OR^{c1}, —NR^{c3}R³, halogen, —C(O)R^{c3}, —C(O)OR^{c3}, —C(O)NR^{c3}R^{c3}, —CN, —NHC(O)R^{c3} and —NHC(O)OR^{c3};
each R^{c3} independently of one another denotes hydrogen or a group selected from among C₁₋₆alkyl, C₂₋₆alkenyl, C₂₋₆alkynyl, C₃₋₆cycloalkyl, C₄₋₆cycloalkenyl, C₁₋₆alkyl-O—C₁₋₆alkyl, (C₁₋₄alkyl)HN—C₁₋₆alkyl, (C₁₋₄alkyl)₂N—C₁₋₆alkyl, C₁₋₆haloalkyl, 4-16 membered heterocyclylalkyl and 3-10 membered heterocyclyl, wherein the heterocyclyl ring in aforementioned groups is optionally substituted by one or more, identical or different C₁₋₆alkyl;
wherein the compounds (I) may optionally also be present in the form of the tautomers, racemates, enantiomers, diastereomers and the mixtures thereof or as the respective salts of all the above-mentioned forms.

2. The compound according to claim 1, wherein
R² denotes C₁₋₆alkyl or phenyl substituted with one or more, identical or different halogen.

3. The compound according to claim 2, wherein
R² is selected from among ethyl, n-propyl, iso-propyl and iso-butyl.

4. The compound according to claim 3, wherein
R² is n-propyl.

5. The compound according to claim 1, wherein
R³ is halogen.

6. The compound according to claim 5, wherein
R³ is fluorine.

7. The compound according to claim 1, wherein
R⁴ is 3-11 membered heterocyclyl optionally substituted by one or more, identical or different R^{a2} and/or R^{b2};
each R^{a2} independently of one another denotes a group optionally substituted by one or more, identical or different R^{b2} and/or R^{c2}, selected from among C₁₋₆alkyl, C₂₋₆alkenyl, C₂₋₆alkynyl, C₁₋₆haloalkyl, C₃₋₆cycloalkyl, C₄₋₆cycloalkenyl and 3-10 membered heterocyclyl;
each R^{b2} is independently selected from among —OR^{c2}, —NR^{c2}R^{c2}, halogen, —C(O)R^{c2}, —C(O)OR^{c2}, —C(O)NR^{c2}R^{c2}, —CN, —NHC(O)R^{c2} and —NHC(O)OR^{c2}, and
each R^{c2} independently of one another denotes hydrogen or a group selected from among C₁₋₆alkyl, C₂₋₆alkenyl, C₂₋₆alkynyl, C₃₋₆cycloalkyl, C₄₋₆cycloalkenyl and 3-10 membered heterocyclyl, wherein this heterocyclyl is optionally substituted by one or more, identical or different substituents selected from among halogen, C₁₋₆alkyl and —C(O)—C₁₋₆alkyl.

8. The compound according to claim 7, wherein
R⁴ is 4-7 membered, nitrogen-containing heterocyclyl optionally substituted by one or more, identical or different R^{a2} and/or R^{b2};
each R^{a2} independently of one another denotes a group optionally substituted by one or more, identical or different R^{b2} and/or R^{c2}, selected from among C₁₋₆alkyl, C₂₋₆alkenyl, C₂₋₆alkynyl, C₁₋₆haloalkyl, C₃₋₆cycloalkyl, C₄₋₆cycloalkenyl and 3-10 membered heterocyclyl;
each R^{b2} is independently selected from among —OR^{c2}, —NR^{c2}R^{c2}, halogen, —C(O)R^{c2}, —C(O)OR^{c2}, —C(O)NR^{c2}R^{c2}, —CN, —NHC(O)R^{c2} and —NHC(O)OR^{c2}, and
each R^{c2} independently of one another denotes hydrogen or a group selected from among C₁₋₆alkyl, C₂₋₆alkenyl, C₂₋₆alkynyl, C₃₋₆cycloalkyl, C₄₋₆cycloalkenyl and 3-10 membered heterocyclyl, wherein this heterocyclyl is optionally substituted by one or more, identical or different substituents selected from among halogen, C₁₋₆alkyl and —C(O)—C₁₋₆alkyl.

9. The compound according to claim 8, wherein
R⁴ is selected from among piperazinyl, piperidinyl, pyrrolidinyl and morpholinyl, all optionally substituted by one or more, identical or different R^{a2} and/or R^{b2};
each R^{a2} independently of one another denotes a group optionally substituted by one or more, identical or different R^{b2} and/or R^{c2}, selected from among C₁₋₆alkyl, C₂₋₆alkenyl, C₂₋₆alkynyl, C₁₋₆haloalkyl, C₃₋₆cycloalkyl, C₄₋₆cycloalkenyl and 3-10 membered heterocyclyl;
each R^{b2} is independently selected from among —OR^{c2}, —NR^{c2}R^{c2}, halogen, —C(O)R^{c2}, —C(O)OR^{c2}, —C(O)NR^{c2}R^{c2}, —CN, —NHC(O)R^{c2} and —NHC(O)OR^{c2}, and
each R^{c2} independently of one another denotes hydrogen or a group selected from among C₁₋₆alkyl, C₂₋₆alkenyl, C₂₋₆alkynyl, C₃₋₆cycloalkyl, C₄₋₆cycloalkenyl and 3-10 membered heterocyclyl, wherein this heterocyclyl is optionally substituted by one or more, identical or different substituents selected from among halogen, C₁₋₆alkyl and —C(O)—C₁₋₆alkyl.

10. The compound according to claim 9, wherein
R⁴ is selected from among piperazinyl, piperidinyl, pyrrolidinyl and morpholinyl, all bound to the pyrimido[5,4-d]pyrimidine ring system via a nitrogen atom and all optionally substituted by one or more, identical or different R^{a2} and/or R^{b2};
each R^{a2} independently of one another denotes a group optionally substituted by one or more, identical or different R^{b2} and/or R^{c2}, selected from among C₁₋₆alkyl, C₂₋₆alkenyl, C₂₋₆alkynyl, C₁₋₆haloalkyl, C₃₋₆cycloalkyl, C₄₋₆cycloalkenyl and 3-10 membered heterocyclyl;
each R^{b2} is independently selected from among —OR^{c2}, —NR^{c2}R^{c2}, halogen, —C(O)R^{c2}, —C(O)OR^{c2}, —C(O)NR^{c2}R^{c2}, —CN, —NHC(O)R^{c2} and —NHC(O)OR^{c2}, and
each R^{c2} independently of one another denotes hydrogen or a group selected from among C₁₋₆alkyl, C₂₋₆alkenyl, C₂₋₆alkynyl, C₃₋₆cycloalkyl, C₄₋₆cycloalkenyl and 3-10 membered heterocyclyl, wherein this heterocyclyl is optionally substituted by one or more, identical or different substituents selected from among halogen, C₁₋₆alkyl and —C(O)—C₁₋₆alkyl.

11. The compound according to claim 7, wherein
each R^{a2} independently of one another denotes a group optionally substituted by one or more, identical or different R^{b2} and/or R^{c2}, selected from among C₁₋₆alkyl, C₃₋₆cycloalkyl and 3-10 membered heterocyclyl;
each R^{b2} is independently selected from among —OR^{c2}, —NR^{c2}R^{c2} and halogen, and
each R^{c2} independently of one another denotes hydrogen or a group selected from among C₁₋₆alkyl, C₃₋₆cycloalkyl and 3-10 membered heterocyclyl, wherein this heterocyclyl is optionally substituted by one or more, identical or different substituents selected from among halogen, C₁₋₆alkyl and —C(O)—C₁₋₆alkyl.

12. The compound according to claim 1, wherein
R⁴ is —NR^{a3}R^{a3};
each R^{a3} independently of one another denotes hydrogen or a group optionally substituted by one or more, identical or different R^{b3} and/or R^{c2}, selected from among C₁₋₆alkyl, C₂₋₆alkenyl, C₂₋₆alkynyl, C₁₋₆haloalkyl, C₃₋₆cycloalkyl, C₄₋₆cycloalkenyl, C₆₋₁₀aryl, 5-10 membered heteroaryl and 3-10 membered heterocyclyl;
each R^{b3} is independently selected from among —OR^{c3}, —NR^{c3}R^{c3}, halogen, —C(O)R^{c3}, —C(O)OR^{c3}, —C(O)NR^{c3}R^{c3}, —CN, —NHC(O)R^{c3} and —NHC(O)OR^{c3};
each R^{c3} independently of one another denotes hydrogen or a group selected from among C₁₋₆alkyl, C₂₋₆alkenyl, C₂₋₆alkynyl, C₃₋₆cycloalkyl, C₄₋₆cycloalkenyl, C₁₋₆alkyl-O—C₁₋₆alkyl, (C₁₋₄alkyl)HN—C₁₋₆alkyl, (C₁₋₄alkyl)₂N—C₁₋₆alkyl, C₁₋₆haloalkyl, 4-16 membered heterocyclylalkyl and 3-10 membered heterocyclyl, wherein the heterocyclyl ring in aforementioned groups is optionally substituted by one or more, identical or different C₁₋₆alkyl;

13. The compound according to claim 1, wherein
R⁴ is —NR⁵R⁶;
R⁵ is hydrogen and
R⁶ is selected from among C₃₋₆cycloalkyl, 3-7 membered heterocyclyl, phenyl and pyridyl, all optionally substituted by one or two, identical or different substituents selected from among C₁₋₆alkyl, C₁₋₆alkyloxy-C₁₋₆alkyl and 3-7 membered heterocyclyl, wherein said last mentioned 3-7 membered heterocyclyl is optionally substituted by C₁₋₆alkyl.

14. The compound according to claim 1, wherein
R⁴ is 5-6 membered, nitrogen-containing heteroaryl, optionally substituted by one, two or three, identical or different C₁₋₄alkyl.

15. The compound according to claim 14, wherein
R⁴ is 5-membered, nitrogen-containing heteroaryl bound to the pyrimido[5,4-d]pyrimidine ring system via a nitrogen atom and optionally substituted by one, two or three, identical or different C₁₋₄alkyl.

16. The compound according to claim 15, wherein
R⁴ is imidazolyl bound to the pyrimido[5,4-d]pyrimidine ring system via a nitrogen atom and optionally substituted by one, two or three, identical or different C₁₋₄alkyl.

17. The compound according to claim 1, wherein
X denotes chlorine.

18. The compound according to claim 1, wherein
X denotes fluorine.

19. A compound—or pharmaceutically acceptable salts thereof—selected from among:

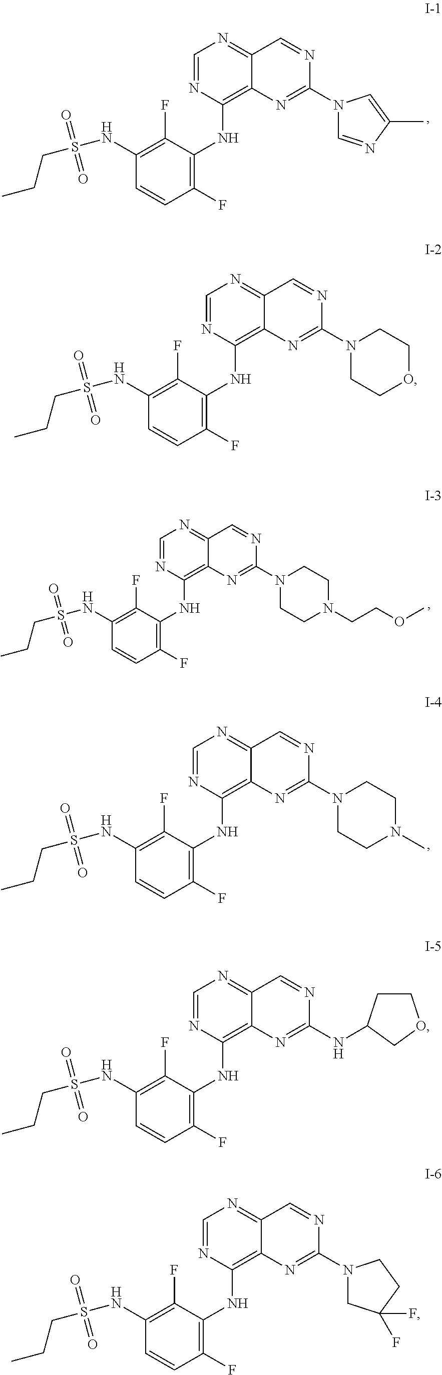

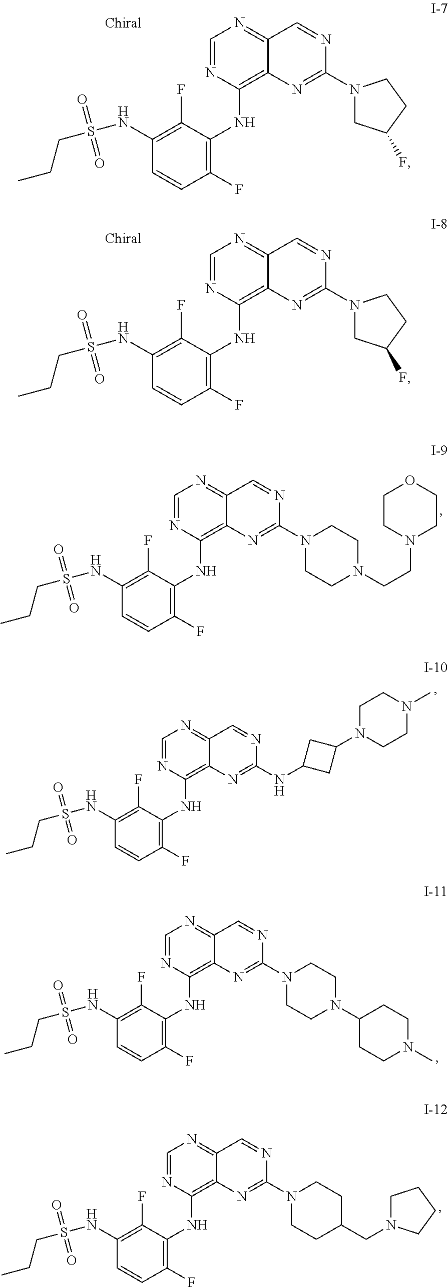

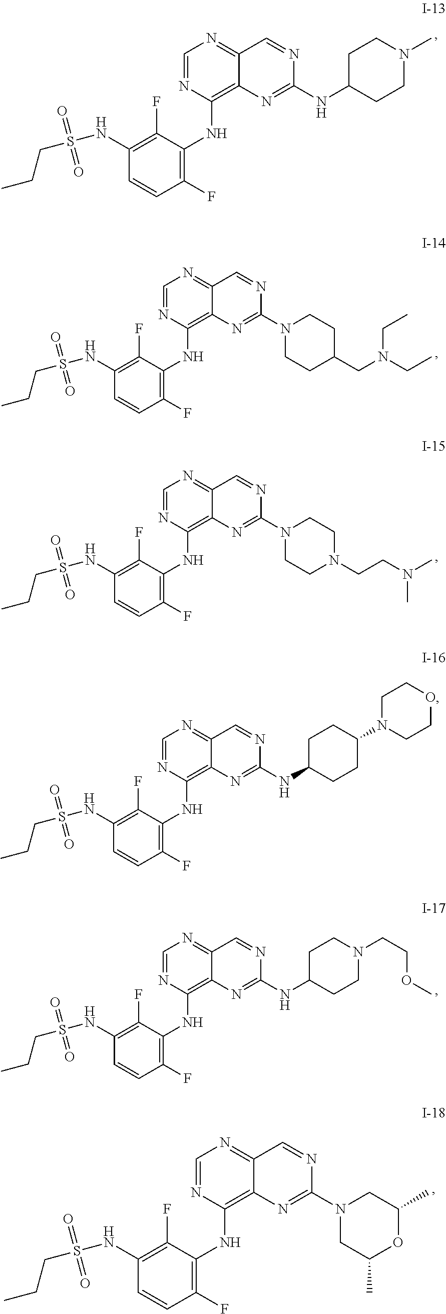

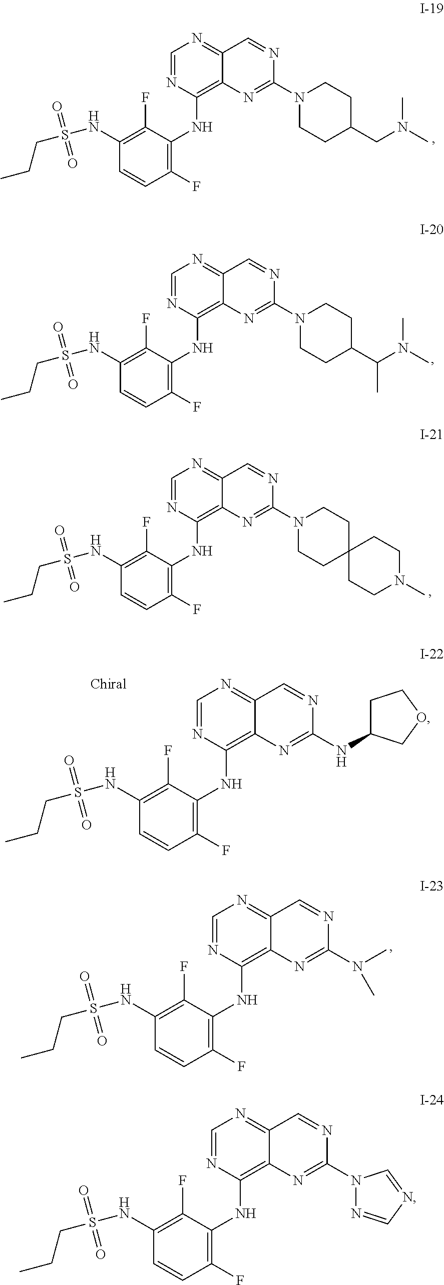

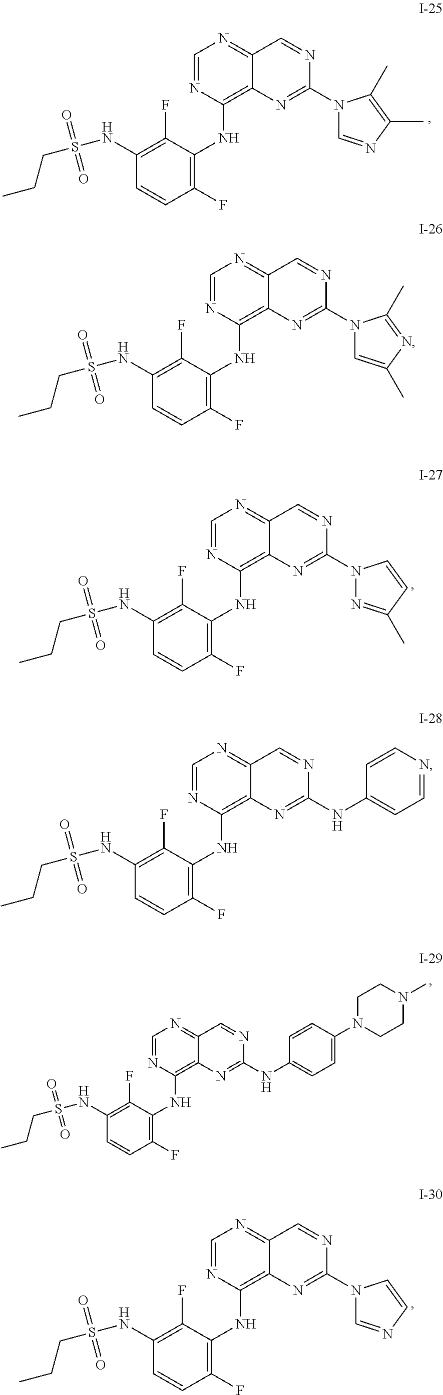

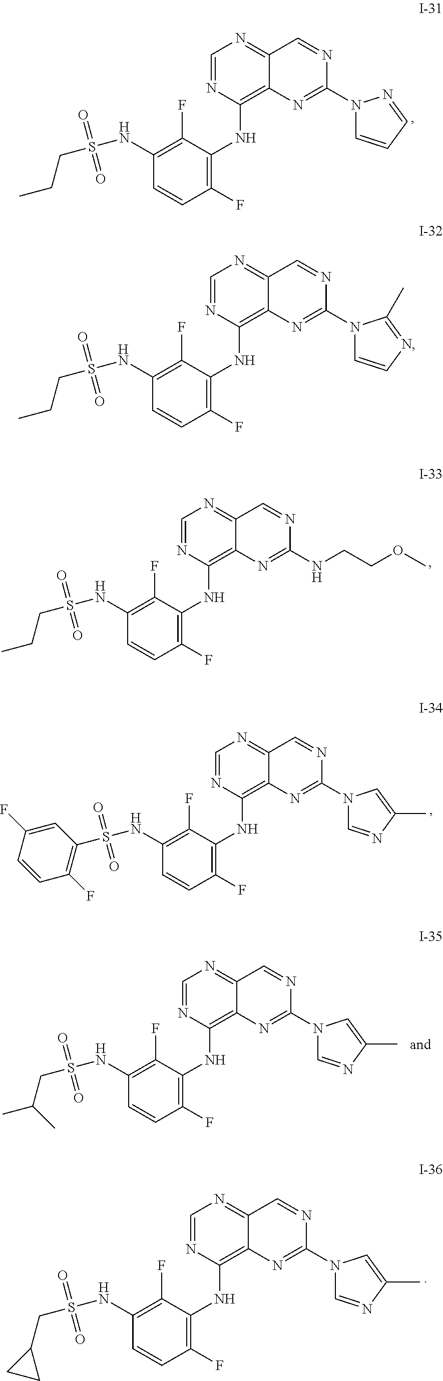

20. The compound according to claim 1 wherein the salt is a pharmaceutically acceptable salt.

21. A method of treating colon carcinomas, melanomas, cancer of the gall bladder or thyroid carcinomas comprising administering to a subject a therapeutically effective amount of a compound according to claim 1.

22. The method according to claim 21 wherein the subject is a human being.

23. A pharmaceutical composition comprising a compound of the formula (I) according to claim 1—or the pharmaceutically acceptable salt thereof—optionally in combination with conventional excipients and/or carriers.

24. The pharmaceutical composition according to claim 23 further comprising at least one other cytostatic or cytotoxic active substance, different from the formula (I).
